(19) United States
(12) Patent Application Publication
(10) Pub. No.: US 2026/0240647 A1
(43) Pub. Date: Aug. 20, 2026

(54) PREPARATION OF EXPANDABLE PROSTHETIC VALVE

(71) Applicant: St. Jude Medical, Cardiology Division, Inc., St. Paul, MN (US)

(72) Inventors: Alec King, Maple Grove, MN (US); Abigail C. Gifford, Woodbury, MN (US); Hans Rieckmann, Minneapolis, MN (US); The M. Tang, Saint Paul, MN (US)

(73) Assignee: St. Jude Medical, Cardiology Division, Inc., St. Paul, MN (US)

(21) Appl. No.: 19/428,199

(22) Filed: Dec. 21, 2025

Related U.S. Application Data
(60) Provisional application No. 63/758,885, filed on Feb. 14, 2025.

(51) Int. Cl. A61F 2/24; A61F 2/95; A61M 25/10

(52) U.S. Cl. CPC A61F 2/2418; A61F 2/2415; A61F 2/2433; A61F 2/9524; A61F 2230/0069; A61M 25/10184

(57) ABSTRACT

There are provided methods and systems for preparing an expandable prosthetic heart valve and assemblies comprising prepared valves. The methods comprise the steps of inserting a support member into the prosthetic heart valve whilst the prosthetic heart valve is in an expanded state, arranging the support member and prosthetic heart valve such that the support member contacts two or more leaflets of the prosthetic heart valve, and crimping the prosthetic heart valve to reduce its external diameter and the external diameter of the support member whilst the support member is maintained in contact with the two or more leaflets during crimping.

### CROSS-REFERENCE TO RELATED APPLICATIONS

The present application claims priority to the filing date of U.S. Provisional Patent Application No. 63/758,885, filed Feb. 14, 2025, the disclosure of which is hereby incorporated by reference herein.

### BACKGROUND

The present disclosure relates to the preparation and crimping of collapsible prosthetic heart valve and delivery systems for collapsible prosthetic heart valves.

Prosthetic heart valves that are collapsible to a relatively small circumferential size can be delivered into a patient less invasively than valves that are not collapsible. For example, a collapsible valve may be delivered into a patient via a tube-like delivery apparatus such as a catheter, a trocar, a laparoscopic instrument, or the like. This collapsibility can avoid the need for a more invasive procedure such as full open-chest, open-heart surgery.

Collapsible prosthetic heart valves typically take the form of a valve structure mounted on a stent. There are two types of stents on which the valve structures are ordinarily mounted: a self-expanding stent and a balloon-expandable stent. To place such valves into a delivery apparatus and ultimately into a patient, the valve must first be collapsed or crimped to reduce its circumferential size.

When a collapsed prosthetic valve has reached the desired implant site in the patient (e.g., at or near the annulus of the patient's heart valve that is to be replaced by the prosthetic valve), the prosthetic valve can be deployed or released from the delivery apparatus and expanded (or re-expanded) to full operating size. For balloon-expandable valves, this generally involves assuring its proper location, and then expanding a balloon positioned within the valve stent. For self-expanding valves, on the other hand, the stent automatically expands as a sheath covering the valve is withdrawn.

### SUMMARY OF INVENTION

Improvements to prosthetic heart valves are sought. In particular, it is desirable to provide prosthetic heart valves with a low-profile in their delivery state. That is the valves have a small diameter—referred to herein interchangeably as the delivery diameter or crimp profile—during delivery such that the effect on a patient is reduced. Furthermore, it is desirable to minimise damage or stress to the leaflets of a prosthetic heart valve during the preparation of the valve for delivery. Such damage can prevent the valves from functioning correctly or reduce the functional lifespan of the valve. The invention offers improvements to these issues.

Various aspects of the present invention are set out in the appended claims.

According to a first aspect of the invention, there is provided a method of preparing an expandable prosthetic heart valve comprising a stent and a plurality of leaflets for delivery, the method comprising the steps of inserting a support member into the prosthetic heart valve whilst the prosthetic heart valve is in an expanded state, arranging the support member and prosthetic heart valve such that the support member contacts two or more leaflets of the plurality of leaflets of the prosthetic heart valve and crimping the prosthetic heart valve to reduce its external diameter and the external diameter of the support member. The support member is maintained in contact with the two or more leaflets during crimping such that the support member applies a frictional or radial force to the two or more leaflets as the external diameter of the prosthetic heart valve is reduced.

By supporting the leaflets of the prosthetic heart valve during the crimping using a support member (e.g. an inflated balloon or a compressible body) undesirable bunching or folding of the leaflets is prevented. The leaflets may be maintained in a uniform and consistent arrangement throughout crimping. This enables the prosthetic heart valve to be crimped to a smaller diameter and reduces the risk of damage to the leaflets during crimping which could affect the lifespan or performance of the prosthetic heart valve. Preferably the support member is configured to contact and support all of the leaflets of the valve during crimping.

As such, the support member is preferably arranged in contact with all leaflets of said plurality of leaflets of the prosthetic heart valve simultaneously when the prosthetic heart valve is in an expanded state and is maintained in contact with all of the leaflets of the valve during the crimping step.

Preferably arranging the support member and prosthetic heart valve such that the support member contacts the two or more leaflets of the prosthetic heart valve comprises arranging the support member to push the leaflets outwards towards the stent. Thus, the support member may apply a radial force to the two or more leaflets that pushes said leaflets outwards towards the stent. The two or more leaflets may be flattened against the stent (or any intervening layer such as a cuff). In this manner the leaflets may be placed in a fully open arrangement. This flattened or fully open arrangement is particularly beneficial as it helps prevent folding or bunching to the leaflets during the subsequent crimping step and promotes a uniform and consistent arrangement of leaflets in the crimped valve. Consequently, the valve may be crimped to a smaller final diameter (the so-called “delivery diameter”) and its lifespan and performance may be improved.

Preferably the support member is configured to restrict movement of the two or more leaflets relative to the stent as the external diameter of the prosthetic heart valve is reduced. Particularly preferably the support member is configured to restrict circumferential and/or longitudinal movement of the two or more leaflets relative to the stent as the external diameter of the prosthetic heart valve is reduced. Therefore, undesirable folding or bunching of the leaflets is restricted. Again, this may help reduce the delivery diameter of the valve and improve its lifespan and performance.

Preferably, arranging the support member and prosthetic heart valve such that the support member contacts the two or more leaflets of the prosthetic heart valve comprises arranging the support member such that its external diameter that is greater than 80% of the internal diameter of the stent when the stent is in its expanded state, preferably greater than 90%, more preferably greater than 95%. As such, the support member may contact the leaflets and push them in an outward direction.

Preferably the support member is a balloon. The balloon is preferably inserted into the prosthetic heart valve while the balloon is in a deflated or partially inflated state. Alternatively the balloon may be inserted into the prosthetic heart valve while the balloon is in a fully inflated state.

The balloon may comprise a compliant balloon material such as polyurethane or silicone. Preferably the balloon has a single wall and has a wall thickness in the range from 0.025 mm to 0.15 mm, preferably from 0.05 mm to 0.1 mm (approximately 0.001 to 0.006 inch, preferably in the range from approximately 0.002 to 0.004 inches). However, other balloons with multiple walls and other wall thicknesses are also possible. The balloon is preferably configured to maintain a cylindrical shape throughout the crimping process.

More preferably, arranging the support member and prosthetic heart valve such that the support member contacts the two or more leaflets of the prosthetic heart valve comprises inflating the balloon to a crimping pressure such that it contacts the two or more leaflets of the prosthetic heart valve, and the method comprises maintaining sufficient internal pressure within the balloon during crimping in order to maintain the balloon in contact with the two or more leaflets.

Preferably, crimping the prosthetic heart valve comprises partially crimping the prosthetic heart valve to reduce its external diameter to a support release diameter, and the method comprises the further steps of removing the support member from inside the partially crimped prosthetic heart valve and crimping the prosthetic heart valve further to reduce its external diameter from the support release diameter to a delivery diameter.

Preferably the balloon is a sacrificial balloon, and wherein crimping the prosthetic heart valve comprises partially crimping the prosthetic heart valve to reduce its external diameter to a support release diameter, and the method comprises the further steps of removing the sacrificial balloon from inside the partially crimped prosthetic heart valve inserting a delivery balloon into the prosthetic heart valve and crimping the prosthetic heart valve further to reduce its external diameter to a delivery diameter.

Preferably removing the sacrificial balloon from inside the partially crimped prosthetic heart valve comprises deflating the sacrificial balloon.

Preferably, the method comprises inflating the delivery balloon such that it contacts two or more leaflets of the plurality of leaflets of the prosthetic heart valve and maintaining sufficient internal pressure within the delivery balloon during crimping in order to maintain the delivery balloon in contact with said two or more leaflets. Preferably the method comprises inflating the delivery balloon such that it contacts all of the leaflets of the plurality of leaflets of the prosthetic heart valve.

Preferably, a pressure within the balloon is maintained at a first predetermined crimping pressure during crimping. Therefore, fluid within the balloon may be released as the diameter of the balloon is reduced during crimping. The first predetermined crimping pressure may be in the range from 0 to 203 kPa above atmospheric pressure, preferably from 0 to 101 kPa above atmospheric pressure.

Preferably, during the step of crimping the prosthetic heart valve further to reduce its external diameter to a delivery diameter. the pressure within the delivery balloon is maintained at a second predetermined crimping pressure. Therefore, fluid within the balloon may be released as the diameter of the balloon is reduced during crimping to maintain the internal pressure at this second predetermined crimping pressure. The second predetermined crimping pressure may be in the range from 0 to 203 kPa above atmospheric pressure, preferably from 0 to 101 kPa above atmospheric pressure.

Alternatively, during one or more of the crimping steps, the pressure within the balloon may be allowed to gradually reduce as the valve is crimped. For instance, the pressure within the balloon may be allowed to reduce gradually such that the balloon is fully deflated as the crimping process ends.

Preferably the pressure within the balloon and/or delivery balloon is controlled using a pressure regulator. Preferably the pressure regulator comprises one or more of: a syringe; manual syringe; a pressure gauge; a pressure relief valve; or a pump.

In particularly preferred examples, the method comprises crimping the prosthetic heart valve to reduce its external diameter to an intermediate diameter while sufficient internal pressure within the balloon or delivery balloon is maintained in order to maintain the delivery balloon in contact with the two or more leaflets, releasing the internal pressure within the balloon or delivery balloon, and crimping the prosthetic heart valve further to reduce its external diameter from the intermediate diameter to a delivery diameter.

Preferably the balloon and prosthetic heart valve are configured such that the crimped prosthetic heart valve may be expanded to its expanded state when the balloon or delivery balloon is inflated to a valve expansion pressure. For example the method may further comprise inflating the balloon or delivery balloon to a valve expansion pressure such that the prosthetic heart valve is expanded to its expanded state. This step may be performed during delivery and/or implantation of the prosthetic heart valve. Preferably, the first predetermined crimping pressure is less than 25% of the valve expansion pressure, preferably less than 10%, more preferably still less than 5%, and/or, the second predetermined crimping pressure is less than 25% of the valve expansion pressure, preferably less than 10%, more preferably still less than 5%. Preferably the valve expansion pressure is in the range from 507 kPa to 1.52 MPa above atmospheric pressure.

Preferably the method comprises inflating the balloon and/or delivery balloon with saline or air. However alternative fluids may also be used.

Preferably the method comprises receiving a balloon comprising a proximal portion, a distal portion and a central portion positioned between the proximal portion and the distal portion, the central portion being configured to receive the prosthetic heart valve in a crimped condition. More preferably, the proximal portion and/or distal portion of the balloon is folded into a pillow (e.g. a proximal or distal pillow) having an equal or larger external diameter than the delivery diameter of the prosthetic heart valve or an equal or larger external diameter than the diameter of an adjacent end of the prosthetic heart valve when the prosthetic heart valve is crimped onto the central portion of the balloon and the balloon is deflated. Preferably the method comprises securing one or more of the pillowed proximal portion and the pillowed distal portion before crimping to prevent the corresponding pillowed portion from expanding when pressure is applied to the balloon. Preferably one or more of the pillowed proximal portion and the pillowed distal portion are secured using a cover, band, sheath, or clamshell during crimping. These pillowed portions help protect the valve during delivery.

In preferred examples the balloon comprises a proximal portion, a distal portion and a central portion positioned between the proximal portion and the distal portion, the central portion being configured to receive the prosthetic heart valve in a crimped condition and the method comprises, subsequent to crimping the prosthetic heart valve to reduce its external diameter, folding the balloon material of the proximal portion to form a pillow having an equal or greater external diameter than the delivery diameter of the prosthetic heart valve or the diameter of an end of the crimped prosthetic heart valve adjacent to said pillow, and/or folding the balloon material of the distal portion to form a pillow having an equal or greater external diameter than the delivery diameter of the prosthetic heart valve or the diameter of an end of the crimped prosthetic heart valve adjacent to said pillow.

In further preferred examples, the balloon comprises a proximal portion, a distal portion and a central portion positioned between the proximal portion and the distal portion, the central portion being configured to receive the prosthetic heart valve in a crimped condition, wherein the wall thickness, wall material, wall material properties or cross-sectional dimensions of the balloon are varied between the central portion and the proximal portion and/or distal portions adjacent to the central portion.

Preferably the central portion of the balloon has a smaller cross-sectional dimension than an adjoining part of the proximal portion and/or an adjoining part of the distal portion, such that the central portion is recessed relative to the proximal portion and/or distal portion.

Preferably the method comprises, subsequent to the step of crimping the prosthetic heart valve to reduce its external diameter, folding a portion of the balloon over at least a part of the proximal edge of the prosthetic heart valve to protect the prosthetic heart valve during delivery, and/or, folding a portion of the balloon over at least a part of the distal edge of the prosthetic heart valve to protect the prosthetic heart valve during delivery.

Preferably the balloon comprises a proximal portion, a distal portion and a central portion positioned between the proximal portion and the distal portion, the central portion being configured to receive the prosthetic heart valve in a crimped condition, wherein the proximal portion comprises a proximal projecting region which has increased cross-sectional dimensions relative to surrounding material of the proximal portion, the proximal projecting region being configured such that the proximal portion may be folded into a pillow or folded over a proximal edge of the prosthetic heart valve, and/or, wherein the distal portion comprises a distal projecting region which has increased internal dimensions relative to surrounding material of the distal portion, the distal projecting region being configured such that the distal portion may be folded into a pillow or folded over a distal edge of the prosthetic heart valve.

In further examples, the support member is a compressible body, and wherein the compressible body has an uncompressed diameter that is greater than 75% of the internal diameter of the stent in its expanded state, preferably greater than 80%, more preferably greater than 90%, more preferably still greater than 95%. For example, the compressible body may have an uncompressed external diameter that is greater or equal to the internal diameter of the expanded valve plus 5 millimetres and/or less than or equal to the internal diameter of the expanded 5 millimetres. In a particularly, preferred example the compressible body may have an internal diameter that is equal to the internal diameter of the expanded valve minus 1 millimetre.

Preferably the compressible body comprises one or more of: foam or rubber. For instance, the compressible body may comprise one or more of a closed cell foam, polyurethane foam, polyethylene foam, polypropylene foam, polyester foam, neoprene rubber foam. As with any of the balloons discussed above, the compressible body is able to maintain the relative position of the leaflets during crimping, but preferably remains sufficiently compressible that it does not significantly inhibit crimping and does not impart so much force during crimping that it damages the leaflets by compression.

Preferably crimping the prosthetic heart valve to reduce its external diameter comprises partially crimping the prosthetic heart valve to reduce its external diameter to a support release diameter, and wherein the method comprises the further steps of removing the compressible body from inside the partially crimped prosthetic heart valve and crimping the prosthetic heart valve further to reduce its external diameter from the support release diameter to a delivery diameter. In preferred examples the method comprises the further steps of inserting a delivery balloon into the prosthetic heart valve when the prosthetic heart valve is in a partially crimped state at which its external diameter is equal to the support release diameter and crimping the prosthetic heart valve further to reduce its external diameter from the support release diameter to a delivery diameter.

The external diameter of the prosthetic heart valve in its expanded state may be the range from 15 to 50 mm, preferably from 18 to 30 mm. The intermediate diameter of the prosthetic heart valve may be in the range from 8 to 20 mm, preferably in the range from 10 to 16 mm. The support release diameter of the prosthetic heart valve may be in the range from 8 to 20 mm, preferably in the range from 10 to 16 mm. The delivery diameter of the prosthetic heart valve may be in the range from 4 to 12 mm, preferably in the range from 5 to 10 mm.

The intermediate diameter may be in the range from 1.1 to 2 times the delivery diameter. The support release diameter may be in the range from 1.1 to 2 times the delivery diameter. The external diameter of the prosthetic heart valve in its expanded state may be in the range from 2 to 5 times the delivery diameter.

According to a further aspect of the invention there may be provided a system comprising an expandable prosthetic heart valve comprising a stent and a plurality of leaflets, a support body configured to be inserted into the prosthetic heart valve when the prosthetic heart valve is in an expanded state and a crimper configured to crimp the prosthetic heart valve to reduce its external diameter. The system is configured such that the support member and prosthetic heart valve can be arranged such that the support member contacts two or more leaflets of the plurality of leaflets of the prosthetic heart valve when the prosthetic heart valve is in an expanded state, and the system is configured to maintain the support member in contact with the two or more leaflets during crimping, such that the support member applies a frictional or radial force to the two or more leaflets as the external diameter of the prosthetic heart valve is reduced.

This system offers corresponding benefits to the methods described above. The system may be further configured to perform any of the optional or preferable steps described above with reference to the preceding aspect of the invention.

Preferably the support member is configured to contact all of the leaflets of the prosthetic heart valve simultaneously when the prosthetic heart valve is in an expanded state and to be maintained in contact with all of the leaflets during crimping.

According to a further aspect of the invention there is provided a method of preparing an expandable prosthetic heart valve comprising a stent and a plurality of leaflets for delivery, the method comprising the steps of inserting a balloon into the prosthetic heart valve whilst the prosthetic heart valve is in an expanded state, wherein the balloon is inserted into the prosthetic heart valve in a deflated or partially inflated state, inflating the balloon such that it contacts two or more leaflets of the plurality of leaflets of the prosthetic heart valve, and crimping the prosthetic heart valve to reduce its external diameter and the external diameter of the balloon, wherein the balloon is maintained in contact with the two or more leaflets during crimping.

Thus, the balloon acts as a support body discussed with reference to the previous aspect of the invention. The method may incorporate any of the optional or preferable features discussed above with reference to the previous aspect of the invention that are applicable to methods involving balloons.

As mentioned, the balloon is inflated to contact the leaflets prior to crimping and may apply an outward force that pushes the two or more leaflets outwards towards the stent. Hence, the leaflets may be flattened against the stent (or any intervening layer such as a cuff). Flattening the leaflets against the stent before crimping promotes a uniform and consistent arrangement of leaflets in the crimped valve. Consequently, the valve may be crimped to a smaller final diameter (the so-called “delivery diameter”) and its lifespan and performance may be improved. Preferably the balloon is configured to contact and support all of the leaflets of the prosthetic heart valve during crimping. Thus the balloon may be placed in contact with all of the leaflets of the valve when the valve is in an expanded state, and maintained in contact with all of the leaflets throughout crimping.

Furthermore, the balloon remains inflated during crimping and continues to contact the two or more leaflets during crimping. As such, the balloon preferably restricts movement of the two or more leaflets relative to the stent as the external diameter of the prosthetic heart valve is reduced. Particularly preferably the balloon is configured to restrict circumferential and/or longitudinal movement of the two or more leaflets relative to the stent as the external diameter of the prosthetic heart valve is reduced. Therefore, undesirable folding or bunching of the leaflets is restricted. This may help reduce the delivery diameter of the valve and improve its lifespan and performance.

Preferably the balloon is configured to contact and support all leaflets of a prosthetic heart valve during crimping.

According to a further aspect of the invention there is provided a system comprising an expandable prosthetic heart valve comprising a stent and a plurality of leaflets for delivery, a balloon configured to be inserted into the prosthetic heart valve when the prosthetic heart valve is in an expanded state and a crimper configured to crimp the prosthetic heart valve to reduce its external diameter. The system is configured such that the balloon may be inserted into the prosthetic heart valve whilst the prosthetic heart valve is in an expanded state and the balloon is in a deflated or partially inflated state. The system is further configured such that the balloon may be inflated in order to contact two or more of the leaflets of the plurality of leaflets of the prosthetic heart valve whilst the balloon is inserted in the expanded prosthetic heart valve, and the system is configured such that the balloon may be maintained in contact with the two or more leaflets during crimping.

This system offers corresponding benefits to the methods described above in relation to the previous aspects. The system may be further configured to perform any of the optional or preferable steps described above with reference to the preceding aspects of the invention.

According to a further aspect of the invention there are provided prosthetic heart valve assemblies manufactured according to the methods discussed above. As such, there are provided a prosthetic heart valve assemblies comprising an expandable prosthetic heart valve comprising a stent and a plurality of leaflets, and a balloon. The expandable prosthetic heart valve is crimped onto the balloon, and the balloon is configured to expand the prosthetic heart valve. Each leaflet comprises an attached edge and a free edge, each leaflet being attached a body of the prosthetic heart valve along the respective attached edge, and the free edges of the leaflets being configured to close and open together following expansion of the prosthetic heart valve. The expandable prosthetic heart valve is crimped according to the methods discussed above.

Preferably the prosthetic heart valve is crimped such that the leaflets are arranged such that, for each leaflet, the distance between a reference point on the attached edge, the reference point being the point on the attached edge which is furthest from the free edge along a central axis of the stent, and a point on the free edge which is nearest to the reference point along a direction parallel to a central axis of the stent is at least 80% of a nominal length of the leaflet, the nominal length of the leaflet being the maximum distance between the attached edge of the leaflet and the free edge of the leaflet along a direction parallel to a centreline of the leaflet when the leaflet is laid flat, preferably at least 90%, more preferably at least 95%. These parameters quantify how bunched the leaflets are along the valve.

For instance, each leaflet may be attached along the respective attached edge to a stent (frame) or cuff (skirt) of the prosthetic valve. In some examples, the distance between the reference point on the attached edge and the point on the free edge which is nearest to the reference point may be measured directly. However, in further examples the attached edge may be obscured in the prepared assembly by a cuff or skirt positioned between the leaflets and the exterior of the valve. In these situations, the distance between the reference point and the free edge of the leaflet may be calculated or determined using on a measurement of the distance between the point on the free edge which is nearest to the reference point on the attached edge and a fixed point on the stent (e.g. the distal or proximal end of the stent, an eyelet of the stent, a specific cell of the stent, or an apex of the frame forming the stent). For instance, the distance between the eyelets of the stent and the position of the reference point on the attached edge may be known. Therefore, the distance between the reference point on the attached edge and the closest point on the free edge may be calculated from a comparison of the distance between the eyelets and the closest point on the free edge and the distance between the eyelets and the reference point on the attached edge. These distances may be measured using a vision system such as a camera or microscope, or using any other suitable measuring device.

Additionally or alternatively, the prosthetic heart valve assembly may be crimped such that the variance, σ², in the position of the free edges of the leaflets is less than or equal to 1.2, preferably less than or equal to 1.0, more preferably less than or equal to 0.9 and more preferably still less than 0.8, the variance in the position of the free edge of the leaflets being calculated as:

$${σ}^{2} = \frac{{\sum }_{i = 1}^{n} {( {x}_{i} - \overline{x} )}^{2}}{N}$$

where σ² is the variance in the position of the free edge of the leaflets, x₁ to xₙ are a plurality of distance measurements along a direction parallel to a central axis of the stent between a plurality of respective points on the free edge of the leaflets and a consistent point on the prosthetic heart valve assembly, \tilde{x} is the mean of the distance measurements, and N is the number of distance measurements in the plurality of distance measurements. The variance σ² provides a measure of how consistently the leaflets of a valve are folded. Highly consistent arrangements with low variance are possible using internal support during crimping, leading to smaller valves with improved performance. Such variances are not thought possible through comparative crimping methods without internal support.

Additionally or alternatively, the prosthetic heart valve assembly may be crimped such that the difference between the average distance between the free edge of each leaflet and the point on the attached edge of each leaflet which is furthest from the free edge along a central axis of the stent and the minimum distance between the free edge of each leaflets and the point on the attached edge of each leaflet which is furthest from the free edge along a central axis of the stent is less than 10%, and preferably less than 8%. These differences quantify the largest amount by which the free edge of the leaflets in a valve have been pushed or wrinkled towards the attached edge of the leaflets during crimping. Valves that have been crimped with internal support offer consistently folded leaflets and a low difference between the shortest height of the leaflet and the average height of the leaflet relative to the belly of the leaflet (the point on the attached edge of each leaflet which is furthest from the free edge along a central axis of the stent) when compared to valves that have been crimped without internal support.

Preferably one or more of the leaflets are interleaved with the material of the balloon around the circumference of the balloon. Additionally, or alternatively, any portions of the leaflets which extend through the cells of the stent extend beyond the struts of the stent by less than 20% of the outer diameter of the crimped prosthetic heart valve, preferably less than 15%, more preferably less than 10%. These distances may be measured on a prepared prosthetic heart valve assembly using a vision system such as a microscope or camera, or using any other suitable measuring device. These parameters quantify how irregularly the leaflets are arranged.

In preferred prosthetic heart valve assemblies at least 75% of the cells in the row of cells furthest from the attached edge of the leaflets receive a single respective longitudinal fold of leaflet material therein, preferably at least 80%, more preferably at least 90%, more preferably still all of the cells in the row of cells furthest from the attached edge of the leaflets receive a single respective longitudinal fold of leaflet material therein, or at least 75% of the cells in the row of cells furthest from the attached edge of the leaflets receive two longitudinal folds of leaflet material therein, preferably at least 80%, more preferably at least 90%, more preferably still all of the cells in the row of cells furthest from the attached edge of the leaflets receive two longitudinal folds of leaflet material therein.

In particularly preferred examples, the stent of the prosthetic heart valve assembly comprises an inflow section and an outflow section, wherein the inflow section comprises a plurality of rows of first cells and the outflow section comprises a row of larger second cells, the second cells defining a larger internal area, height and/or width than the first cells. Additionally, at least 75% of the cells in the outflow-most row of first cells of the inflow section (the row of cells in the inflow section furthest from the attached edge of the leaflets) receive a single respective longitudinal fold of leaflet material therein, preferably at least 80%, more preferably at least 90%, and/or at least 75% of the second cells in the outflow section receive two longitudinal folds of leaflet material therein, preferably at least 80%, more preferably at least 90%.

Assemblies with these arrangements of leaflets cannot be easily manufactured using methods where a prosthetic heart valve is crimped without internal support—e.g. crimped over a deflated balloon as is typical. Using these pre-existing approaches, the leaflets tend to move significantly relative to the stent and bunch in an axial direction of the stent, to not be interleaved with the deflated balloon and to form large “fins” or “flaps” of folded leaflet material that projects large distances through cells in the stent of the valve. When compared to corresponding assemblies manufactured with conventional approaches, the assembly allows for the final delivery diameter of the prosthetic heart valve to be reduced and offers increased valve performance and lifespan. The improvements in consistency of leaflet arrangement are particularly apparent when observing series of prosthetic heart valves and prosthetic heart valve assemblies prepared according to the methods described in reference to the previous aspects of the invention. A series of sequentially or consecutively prepared prosthetic heart valves (or a sample of said series) that have been crimped using internal support body may have a mean of the variances, σ⁶, in the position of the respective free edges of the leaflets in each valve that is less than 1.0, preferably less than 0.9 and more preferably less than 0.8. Whereas, the average of the variances σ⁶ in the position of the respective free edges of the leaflets across a series of valves and valve assemblies crimped without internal support is typically greater than 1.5.

### BRIEF DESCRIPTION OF DRAWINGS

Embodiments in accordance with the present disclosure will now be described with reference to the accompanying drawings, in which:

FIG. 1 is a perspective view of an example of a prosthetic heart valve;

FIG. 2 is a front view of an example of a section of the frame of the prosthetic heart valve of FIG. 1, as if cut longitudinally and laid flat on a table;

FIG. 3 is a front view of an example of a prosthetic leaflet of the prosthetic heart valve of FIG. 1, as if laid flat on a table;

FIG. 4 is a top view of the prosthetic heart valve of FIG. 1 mounted on an example of a portion of a delivery system;

FIG. 5 is an enlarged view of the handle of the delivery system shown in FIG. 4;

FIG. 6 is an enlarged view of a distal end of the delivery system shown in FIG. 4;

FIG. 7 is a top view of an example of a balloon catheter when the balloon is inflated;

FIG. 8 is a top view of an example of an inflation system for use with a delivery system similar to that shown in FIG. 4;

FIG. 9 is a side view of the inflation system of FIG. 8;

FIG. 10 is a perspective view of a connection between the inflation system of FIGS. 8 to 9 and the handle of the delivery system of FIG. 4;

FIG. 11 is a flowchart showing exemplary steps in a procedure to implant the prosthetic heart valve of FIG. 1 into a patient using the delivery system of FIG. 4;

FIG. 12A is a side view of an example of a mechanical crimper suitable for preparing prosthetic heart valves in accordance with aspects of the disclosure;

FIG. 12B is a perspective view of the mechanical crimper of FIG. 12A with certain components being shown in phantom;

FIGS. 13a to 13e show in schematic cross section, sequential steps of a method of preparing an expandable valve for delivery in accordance with the invention;

FIGS. 14a to 14e show in schematic cross section, sequential steps of a further method of preparing an expandable valve for delivery in accordance with the invention;

FIG. 15 shows in schematic cross section a system in accordance with the invention;

FIGS. 16a to 16d show in schematic cross section, steps of further methods of preparing an expandable valve for delivery in accordance with the invention;

FIG. 17 shows in schematic cross section, a balloon suitable for use in methods and systems in accordance with the invention;

FIG. 18 is a flowchart showing exemplary steps in a method of preparing an expandable valve for delivery in accordance with the invention;

FIG. 19 is a flowchart showing exemplary steps in a method of preparing an expandable valve for delivery in accordance with the invention;

FIGS. 20a to 20g show in schematic cross section, sequential steps of a further method of preparing an expandable valve for delivery in accordance with the invention;

FIG. 21a shows a prosthetic heart valve assembly;

FIG. 21b shows a prosthetic heart valve assembly in accordance with the invention;

FIG. 22a is a front view of an example of a prosthetic leaflet of a prosthetic heart valve as if prepared in accordance with methods of the invention;

FIG. 22b is a front view of an example of a prosthetic leaflet of a prosthetic heart valve as if prepared in accordance with comparative methods.

### DETAILED DESCRIPTION

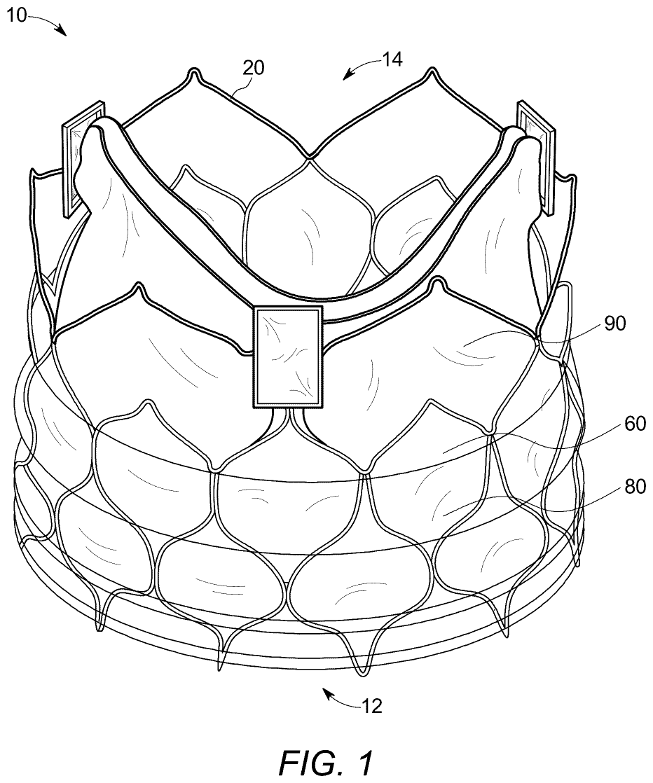

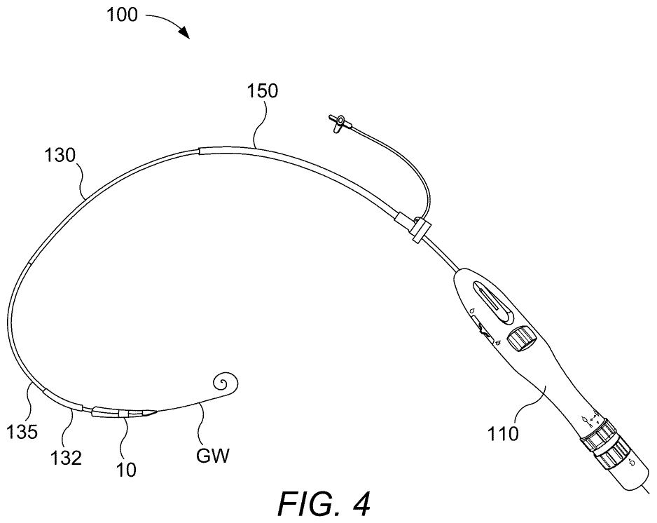

The present disclosure relates to methods, systems and assemblies for preparing a balloon expandable prosthetic heart valve for delivery using a minimally invasive delivery device. An example of a suitable balloon expandable prosthetic heart valve is illustrated in FIG. 1. Whilst an exemplary minimally invasive delivery device is shown in FIG. 4.

In particular, the present disclosure relates to methods, systems and assemblies for crimping (or “collapsing”) a balloon expandable prosthetic heart valve before delivery. Crimping reduces the diameter of the prosthetic heart valves such that they may be easily and safely delivered to a desired implant site. According to the present disclosure, a support member such as an inflated balloon or compressible body may be inserted within a prosthetic heart valve whilst the valve is in an expanded state and arranged to contact the leaflets of the prosthetic heart valve. The support member preferably applies a radial force to the leaflets and preferably this radial force pushes the leaflets outwards towards the surrounding stent. The leaflets may consequently be placed in a fully open position in which they are flattened against the stent, or against any intervening layer between the leaflets and stent. This is a particularly uniform, consistent arrangement for the leaflets. The valve is subsequently crimped whilst the support member contacts the leaflets. As such, the support member continues to provide an outward and/or frictional force to the leaflets and restricts the movement of the leaflets relative to the stent during crimping. Hence, the leaflets may be supported in their flattened, fully open position as the external diameter of the prosthetic heart valve is reduced. This restricts the leaflets from bunching or folding irregularly, and especially restricts bunching or folding in the axial direction through the valve.

The resulting crimped prosthetic heart valves have a more compact, uniform and consistent arrangement of leaflets when compared to valves obtained through conventional approaches in which the prosthetic heart valves are crimped over a balloon whilst the balloon is deflated and whilst leaflets are unsupported. Using the methods and systems discussed herein, prosthetic heart valves may be crimped to a smaller and more consistent delivery diameter (“crimp profile”) than these existing approaches. Furthermore, stress or damage to the leaflets during crimping is reduced. As such, the performance and lifespan of the installed valves can be improved.

As used herein, the term “inflow end” when used in connection with a prosthetic heart valve refers to the end of the prosthetic valve into which blood first enters when the prosthetic valve is implanted in an intended position and orientation, while the term “outflow end” refers to the end of the prosthetic valve where blood exits when the prosthetic valve is implanted in the intended position and orientation. Thus, for a prosthetic aortic valve, the inflow end is the end nearer the left ventricle while the outflow end is the end nearer the aorta. The intended position and orientation are used for the convenience of describing valves disclosed herein. However, it should be noted that the use of the valve is not limited to the intended position and orientation but may be deployed in any type of lumen or passageway. For example, although prosthetic heart valves are described herein as prosthetic aortic valves, those same or similar structures and features can be employed in other heart valves, such as the pulmonary valve, the mitral valve, or the tricuspid valve. Further, the term “proximal,” when used in connection with a delivery device or system, refers to a position relatively close to the user of that device or system when it is being used as intended, while the term “distal” refers to a position relatively farfrom the user of the device. In other words, the leading end of a delivery device or system is positioned distal to the trailing end of the delivery device or system, when the delivery device is being used as intended. As used herein, the terms “substantially,” “generally,” “approximately,” and “about” are intended to mean that slight deviations from absolute are included within the scope of the term so modified. As used herein, the prosthetic heart valves may assume an “expanded state” and a “collapsed state,” which refer to the relative radial size of the stent.

Collapsible and expandable prosthetic heart valves typically take the form of a one-way valve structure (often referred to as a valve assembly) mounted within an expandable frame (the terms “stent” and “frame” may be used interchangeably herein). In general, these collapsible and expandable heart valves include a self-expanding, mechanically-expandable, or balloon-expandable frame, often made of nitinol or another shape-memory metal or metal alloy (for self-expanding frames) or steel or cobalt chromium (for balloon-expandable frames). The one-way valve assembly mounted to/within the stent includes one or more leaflets and may also include a cuff or skirt. The cuff may be disposed on the stent's interior or luminal surface, its exterior or abluminal surface, and/or on both surfaces. A cuff helps to ensure that blood does not just flow around the valve leaflets if the valve or valve assembly is not optimally seated in a valve annulus. A cuff, or a portion of a cuff disposed on the exterior of the stent, can help prevent leakage around the outside of the valve (the latter known as paravalvular or “PV” leakage).

Balloon expandable valves are typically delivered to the native annulus while collapsed (or “crimped”) onto a balloon (or “delivery balloon”) of a balloon catheter, with the collapsed valve being either covered or uncovered by an overlying sheath. Once the crimped prosthetic heart valve is positioned within the annulus of the native heart valve that is being replaced, the balloon is inflated to force the balloon-expandable valve to transition from the collapsed or crimped condition into an expanded or deployed condition, with the prosthetic heart valve tending to remain in the shape into which it is expanded by the balloon. Typically, when the position of the collapsed prosthetic heart valve is determined to be in the desired position relative to the native annulus (e.g. via visualization under fluoroscopy), a fluid (typically a liquid although gas could be used as well) such as saline is pushed via a syringe (manually, automatically, or semi-automatically) through the balloon catheter to cause the balloon to begin to fill and expand, and thus cause the overlying prosthetic heart valve to expand into the native annulus.

FIG. 1 is a perspective view of one example of a prosthetic heart valve 10. Prosthetic heart valve 10 may be a balloon-expandable prosthetic aortic valve, although in other examples it may be a self-expandable or mechanically-expandable prosthetic heart valve, intended for replacing a native aortic valve or another native heart valve. Prosthetic heart valve 10 is shown in an expanded condition in FIG. 1. Prosthetic heart valve 10 may extend between an inflow end 12 and an outflow end 14. Prosthetic heart valve 10 may include a collapsible and expandable frame (stent) 20, an inner cuff or skirt 60, an outer cuff or skirt 80, and a plurality of prosthetic leaflets 90. As should be clear below, prosthetic heart valve 10 is merely one example of a prosthetic heart valve, and other examples of prosthetic heart valves may be suitable for use with the concepts described below.

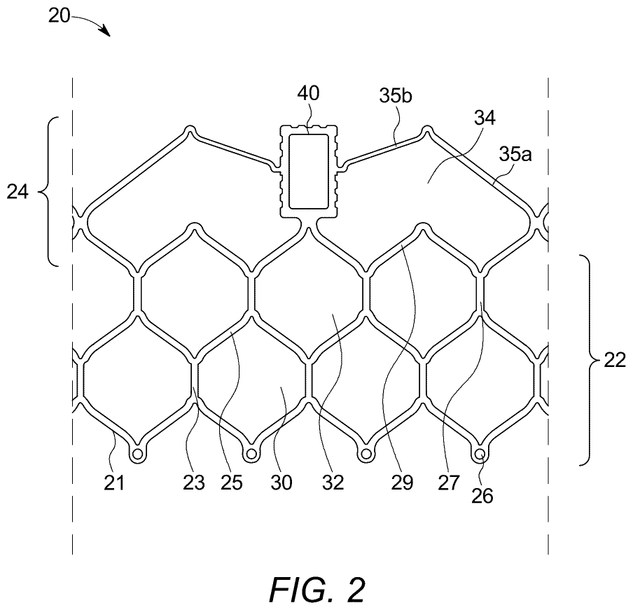

FIG. 2 is a front view of an example of a section of the frame 20 of prosthetic heart valve 10, as if cut longitudinally and laid flat on a table. The section of frame 20 in FIG. 2 may represent approximately one-third of a complete frame, particularly if frame 20 is used in conjunction with a three-leaflet prosthetic heart valve. In the illustrated example, frame 20 is a balloon-expandable stent and may be formed of stainless steel or cobalt-chromium, and which may include additional materials such as nickel and/or molybdenum. However, in some embodiments the stent may be formed of a shape memory material such as nitinol or the like. The frame 20, when provided as a balloon-expandable frame, is configured to collapse upon being crimped to a smaller diameter and/or expand upon being forced open, for example via a balloon within the frame expanding, and the frame will substantially maintain the shape to which it is modified when at rest.

Frame 20 may include an inflow section 22 and an outflow section 24. The inflow section 22 may also be referred to as the annulus section. In one example, the inflow section 22 includes a plurality of rows of generally hexagon-shaped cells. For example, the inflow section 22 may include an inflow-most row of hexagon-shaped cells 30 and an outflow-most row of hexagon-shaped cells 32. The inflow-most row of hexagonal cells 30 may be formed of a first circumferential row of angled or zig-zag struts 21, a second circumferential row of angled or zig-zag struts 25, and a plurality of axial struts 23 that connect the two rows. In other words, each inflow-most hexagonal cell 30 may be formed by two angled struts 21 that form an apex pointing in the inflow direction, two angled struts 25 that form an apex pointing in the outflow direction, and two axial struts that connect the two angled struts 21 to two corresponding angled struts 25. The outflow-most row of hexagonal cells 32 may be formed of the second circumferential row of angled or zig-zag struts 25, a third circumferential row of angled or zig-zag struts 29, and a plurality of axial struts 27 that connect the two rows. In other words, each outflow-most hexagonal cell 32 may be formed by two angled struts 25 that form an apex pointing in the inflow direction, two angled struts 29 that form an apex pointing in the outflow direction, and two axial struts that connect the two angled struts 27 to two corresponding angled struts 29. It should be understood that although the term “outflow-most” is used in connection with hexagonal cells 32, additional frame structure, described in more detail below, is still provided in the outflow direction relative to the outflow-most row of hexagonal cells 32.

In the illustrated embodiment, assuming that frame 20 is for use with a three-leaflet valve and thus the section shown in FIG. 2 represents about one-third of the frame 20, each row of cells 30, 32 includes twelve individual cells. However, it should be understood that more or fewer than twelve cells may be provided per row of cells. Further, the inflow or annulus section 22 may include more or fewer than two rows of cells. Still further, although cells 30, 32 are shown as being hexagonal, the some or all of the cells of the inflow section 22 may have other shapes, such as diamond-shaped, chevron-shaped, or other suitable shapes. In the illustrated embodiment, every cell 30 in the first row is structurally similar or identical to every other cell 30 in the first row, every cell 32 in the second row is structurally similar or identical to every other cell 32 in the second row, and every cell 30 in the first row is structurally similar or identical (excluding the aperture 26) to every cell 32 in the second row. However, in other examples, the cells in each row are not identical to every other cell in the same row or in other rows.

An inflow apex of each hexagonal cell 30 may include an aperture 26 (also termed an eyelet) formed therein, which may accept sutures or similar features which may help couple other elements, such as an inner cuff 60, outer cuff 80, and/or prosthetic leaflets 90, to the frame 20. However, in some examples, one or more or all of the apertures 26 may be omitted.

Still referring to FIG. 2, the outflow section 24 of the frame 20 (also referred to as the hybrid section) may include larger cells 34 that have generally asymmetric shapes. For example, the lower or inflow part of the larger cells 34 may be defined by the two upper struts 29 of a cell 32, and one upper strut 29 of each of the two adjacent cells 32. In other words, the lower end of each larger cell 34 may be formed by a group of four consecutive upper struts 29 of three circumferentially adjacent cells 32. The tops of the larger cells 34 may each be defined by two linking struts 35a, 35b. The first linking strut 35a may couple to a top or outflow apex of a cell 32 and extend upwards at an angle toward a commissure attachment feature (“CAF”) 40. The second linking strut 35b may extend from an end of the first linking strut 35a back downwardly at an angle and connect directly to the CAF 40. To the extent that the larger cells 34 include sides, a first side is defined by a portion of the CAF 40, and a second side is defined by the connection between first linking strut 35a and the corresponding upper strut 29 of the cell 32 attached to the first linking strut 35a.

The CAF 40 may generally serve as an attachment site for leaflet commissures (e.g. where two prosthetic leaflets 90 join each other) to be coupled to the frame 20. In the illustrated example, the CAF 40 is generally rectangular and has a longer axial length than circumferential width. The CAF 40 may define an interior open rectangular space. The struts that form CAF 40 may be generally smooth on the surface defining the open rectangular space, but some or all of the struts may have one or more suture notches on the opposite surfaces. For example, in the illustrated example, CAF 40 includes two side struts (on the longer side of the rectangle) and one top (or outflow) strut that all include alternating projections and notches on their exterior facing surfaces. These projections and notches may help maintain the position of one or more sutures that wrap around these struts. These sutures may directly couple the prosthetic leaflets 90 to the frame 20, and/or may directly couple an intermediate sheet of material (e.g., fabric or tissue) to the CAF 40, with the prosthetic leaflets 90 being directly coupled to that intermediate sheet of material. In some embodiments, tabs or ends of the prosthetic leaflets 90 may be pulled through the opening of the CAF 40, but in other embodiments the prosthetic leaflets 90 may remain mostly or entirely within the inner diameter of the frame 20. It should be understood that balloon-expandable frames are typically formed of metal or metal alloys that are very stiff, particularly in comparison to self-expanding frames. At least in part because of this stiffness, although the prosthetic leaflets 90 may be sutured or otherwise directly coupled to the frame at the CAFs 40, it may be preferable that most or all of the remaining portions of the prosthetic leaflets 90 are not attached directly to the frame 20, but are rather attached directly to an inner skirt 60, which in turn is directly connected to the frame 20. Further, it should be understood that other shapes and configurations of CAFs 40 may be appropriate. For example, various other suitable configurations of frames and CAFs are described in greater detail in U.S. patent application Ser. No. 18/810,994, filed Aug. 21, 2024 and titled “TAVI Deployment Accuracy—Stent Frame Improvements,” the disclosure of which is hereby incorporated by reference herein.

With the example described above, frame 20 includes two rows of hexagon-shaped cells 30, 32, and a single row of larger cells 34. In a three-leaflet embodiment of a prosthetic heart valve that incorporates frame 20, each row of hexagon-shaped cells 30, 32 includes twelve cells, while the row of larger cells includes six larger cells 34. As should be understood, the area defined by each individual cell 30, 32 is significantly smaller than the area defined by each larger cell 34 when the frame 20 is expanded. There is also significantly more structure (e.g., struts) that create each row of individual cells 30, 32 than structure that creates the row of larger cells 34.

One consequence of the above-described configuration is that the inflow section 22 has a higher cell density than the outflow section 24. In other words, the total numbers of cells, as well as the number of cells per row of cells, is greater in the inflow section 22 compared to the outflow section 24. The configuration of frame 20 described above may also result in the inflow section 22 being generally stiffer than the outflow section 24 and/or more radial force being required to expand the inflow section 22 compared to the outflow section 24, despite the fact that the frame 20 may be formed of the same metal or metal alloy throughout. This increased rigidity or stiffness of the inflow section 22 may assist with anchoring the frame 20, for example after balloon expansion, into the native heart valve annulus. The larger cells 34 in the outflow section 24 may assist in providing clearance to the coronary arteries after implantation of the prosthetic heart valve 10. For example, after implantation, one or more coronary ostia may be positioned above the frame 20, for example above the valley where two adjacent larger cells 34 meet (about halfway between a pair of circumferentially adjacent CAFs 40). Otherwise, one or more coronary ostia may be positioned in alignment with part of the large interior area of a larger cell 34 after implantation. Either way, blood flow to the coronary arteries is not obstructed, and a further procedure that utilizes the coronary arteries (e.g. coronary artery stenting) will not be obstructed by material of the frame 20. Still further, the lower rigidity of the frame 20 in the outflow section 24 may cause the outflow section 24 to preferentially foreshorten during expansion, with the inflow section 22 undergoing a relatively smaller amount of axial foreshortening. This may be desirable because, as the prosthetic heart valve 10 expands, the position of the inflow end of the frame 20 may remain substantially constant relative to the native valve annulus, which may make the deployment of the prosthetic heart valve 10 more precise. This may be, for example, because the inflow end of the frame 20 is typically used to gauge proper alignment with the native valve annulus prior to deployment, so axial movement of the inflow end of the frame 20 relative to the native valve annulus during deployment may make precise placement more difficult.

Referring back to FIG. 1, the prosthetic heart valve 10 may include an inner skirt 60 mounted to the interior surface of frame 20. The inner skirt 60 may be formed of tissue, such as pericardium, although other types of tissue may be suitable. In the illustrated example, the inner skirt 60 is formed of a woven synthetic fabric, such as polyethylene terephthalate (“PET”) or polytetrafluoroethylene (“PTFE”), although other fabrics may be suitable, including fabrics other than woven fabrics. In some examples, the inner skirt 60 has straight or zig-zag shaped inflow and outflow ends that generally follow the contours of the cells 30, 32 of the inflow section 22 of frame 20. Preferably, inner skirt 60 is sutured to the frame 20 along the struts that form cells 30, 32. If apertures 26 are included, inner skirt 60 may also be coupled to frame 20 via sutures passing through apertures 26. Preferably, the inner skirt 60 does not cover (or does not cover significant portions of) the larger cells 34. The inner skirt 60 may be coupled to the frame 20 via mechanisms other than sutures, including for example ultrasonic welding or adhesives. Further, the inner skirt 60 may have shapes other than that shown, and need not have a zig-zag inflow or outflow end, and need not cover every cell in the inflow section 22. In fact, in some examples, the inner skirt 60 may be omitted entirely, with the outer skirt 80 (described in greater detail below) being the only skirt used with prosthetic heart valve 10. If the inner skirt 60 is provided, it may assist with sealing the prosthetic heart valve 10 within the heart, as well as serving as a mounting structure for the prosthetic leaflets 90 (described in greater detail below) within the frame 20.

Still referring to FIG. 1, the prosthetic heart valve 10 may include an outer skirt 60 mounted to the exterior surface of frame 20. The outer skirt 80 may be formed of tissue, such as pericardium, although other types of tissue may be suitable. In the illustrated example, the outer skirt 80 is formed of a woven synthetic fabric, such as PET or PTFE, although other fabrics may be suitable, including fabrics other than woven fabrics. In some examples, the outer skirt 80 has straight or zig-zag inflow end. Preferably, outer skirt 80 is sutured to the frame 20 and/or inner skirt 60 along the inflow edge of the outer skirt 80. If apertures 26 are included, outer skirt 80 may also be coupled to frame 20 via sutures passing through apertures 26. The outer skirt 80 may include a plurality of folds or pleats, such a circumferentially extending folds or pleats. The folds or pleats may be formed in the outer skirt 80 via heat setting, for example by placing the outer skirt 80 within a mold that forces the outer skirt 80 to form folds of pleats, and the outer skirt 80 may be treated with heat so that the outer skirt 80 tends to maintain folds or pleats in the absence of applied forces. The outflow edge of outer skirt 80 may be coupled to the frame 20 at selected, spaced apart locations around the circumference of the frame 20. In some embodiments, the outflow edge of outer skirt 80 may be connected to the inner skirt 60 along a substantially continuous suture line. Some or all of the outer skirt 80 between its inflow and outflow edges may remain not directly couples to the frame 20 or inner skirt 60. Preferably, the outer skirt 80 does not cover (or does not cover significant portions of) the larger cells 34. In use, the outer skirt 80 may directly contact the interior surface of the native heart valve annulus to assist with sealing, including sealing against PV leak. If folds or pleats are included with the outer skirt 80, the additional material of the folds or pleats may help further mitigate PV leak. However, it should be understood that the folds or pleats may be omitted from outer skirt 80, and the outer skirt 80 may have shapes other than that shown. In fact, in some examples, the outer skirt 80 may be omitted entirely, with the inner skirt 60 being the only skirt used with prosthetic heart valve 10. If the inner skirt 60 is omitted, the prosthetic leaflets 90 may be attached directly to the frame 20 and/or directly to the outer skirt 80.

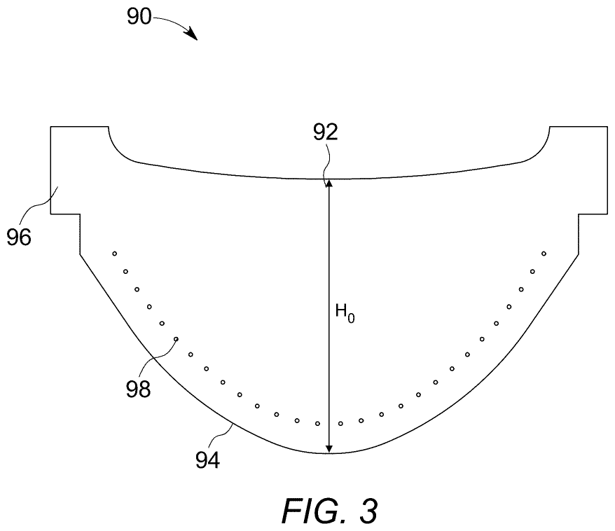

FIG. 3 is a front view of a prosthetic leaflet 90, as if laid flat on a table. In the illustrated example of prosthetic heart valve 10, a total of three prosthetic leaflets 90 are provided, although it should be understood that more or fewer than three prosthetic leaflets may be provided in other example of prosthetic heart valves. The prosthetic leaflet 90 may be formed of a synthetic material, such a polymer sheet or woven fabric, or a biological material, such a bovine or porcine pericardial tissue. However, other materials may be suitable. In one example, the prosthetic leaflet 90 is formed to have a concave free edge 92 configured to cooperate with the free edges of the other leaflets to help provide the one-way valve functionality. The prosthetic leaflet 90 may include an attached edge 94 which is attached (e.g., via suturing) to other bodies or structures of the prosthetic heart valve 10. For example, the attached edge 94 may be coupled directly to the inner skirt 60, directly to the frame (stent) 20, and/or directly to the outer skirt 80. It may be preferable that the attached edge 94 is coupled directly only to the inner skirt 60, which may help reduce stresses on the prosthetic leaflet 90 compared to if the attached edge 94 were coupled directly to the frame 20. In some embodiments, a plurality of holes 98 may be formed along the attached edge 94 (or a spaced distance therefrom), for example via lasers. If included, the holes 98 may be used to receive sutures therethrough, which may make it easier to couple the prosthetic leaflet 90 to the inner skirt 60 during manufacturing. For example, the holes 98 may serve as guides if suturing is performed manually, and if the positions of the holes 98 are controlled via the use of layers, the holes 98 may be consistently placed among different prosthetic leaflets 90 to reduce variability between different prosthetic leaflets 90. Leaflet tabs 96 may be provided at the junctions between the free edge 92 and the attached edge 94. Each leaflet tab 96 may be joined to a leaflet tab of an adjacent prosthetic leaflet to form prosthetic leaflet commissures, which may be coupled to the frame 20 via CAFs 40. The prosthetic leaflet 90 has a nominal length H₀ (also referred to as a nominal height) defined as the maximum distance between the free edge 92 and the attached edge 94 parallel to or along a centreline of the leaflet 90. As shown, this is the distance between the free edge 92 and the extreme point at the belly of the attached edge 94. Following assembly, the centreline of the leaflet 90 is typically aligned with the centerlines of a stent and valve into which the leaflet is secured.

The prosthetic heart valve 10 may be delivered via any suitable transvascular route, for example transapically or transfemorally. Generally, transapical delivery utilizes a relatively stiff catheter that pierces the apex of the left ventricle through the chest of the patient, inflicting a relatively higher degree of trauma compared to transfemoral delivery. In a transfemoral delivery, a delivery device housing or supporting the valve is inserted through the femoral artery and advanced against the flow of blood to the left ventricle. In either method of delivery, the valve may first be crimped (collapsed) over an expandable balloon (a delivery balloon). The balloon may be coupled to or disposed within a delivery system, which may transport the valve through the body and heart to reach the aortic valve, with the valve being disposed over the balloon (and, in some circumstances, under an overlying sheath). Upon arrival at or adjacent to the aortic valve, a surgeon or operator of the delivery system may align the prosthetic valve as desired within the native valve annulus while the prosthetic valve is collapsed over the balloon. When the desired alignment is achieved, the overlying sheath, if included, may be withdrawn (or advanced) to uncover the prosthetic valve, and the balloon may then be expanded causing the prosthetic valve to expand in the radial direction, with at least a portion of the prosthetic valve foreshortening in the axial direction.

FIG. 4 illustrates one example of a delivery system 100, with the prosthetic heart valve 10 crimped over a balloon on a distal end of the delivery system 100. Although delivery system 100 and various components thereof are described below, it should be understood that delivery system 100 is merely one example of a balloon catheter that may be appropriate for use in delivering and deploying prosthetic heart valve 10.

In some examples, delivery system 100 includes a handle 110 and a delivery catheter 130 extending distally from the handle 110. An introducer 150 may be provided with the delivery system 100. Introducer 150 may be an integrated or captive introducer, although in other embodiments introducer 150 may be a non-integrated or non-captive introducer. In some examples, the introducer 150 may be an expandable introducer, including for example an introducer that expands locally as a large diameter component passes through the introducer, with the introducer returning to a smaller diameter once the large diameter components passes through the introducer. In other examples, the introducer 150 is a non-expandable introducer.

A guidewire GW may be provided that extends through the interior of all components of the delivery system 100, from the proximal end of the handle 110 through the atraumatic distal tip 138 of the delivery catheter 130. The guidewire GW may be introduced into the patient to the desired location, and the delivery system 100 may be introduced over the guidewire GW to help guide the delivery catheter 130 through the patient's vasculature over the guidewire GW.

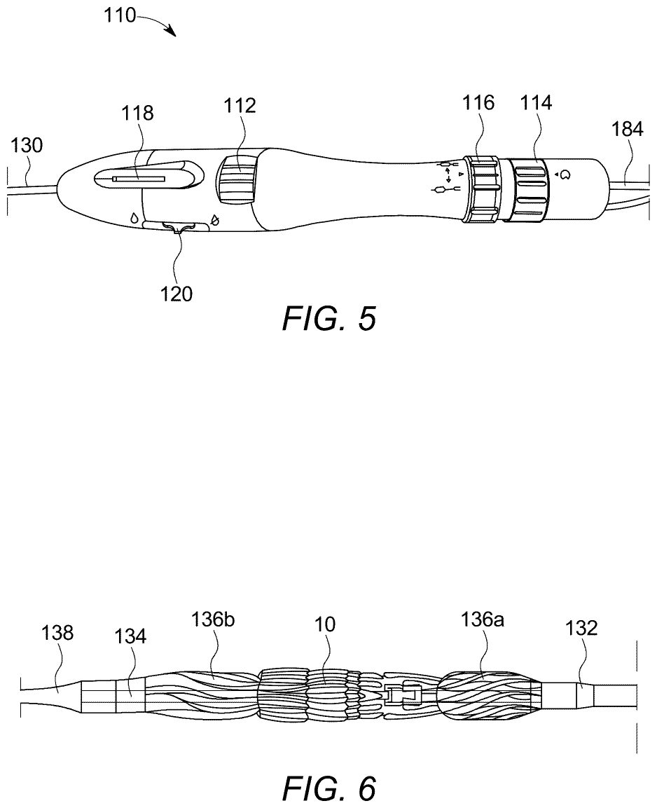

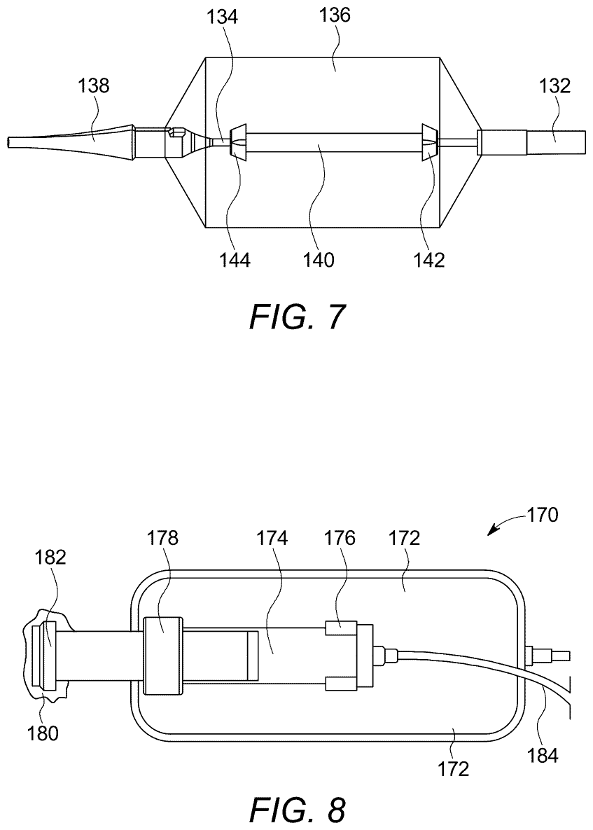

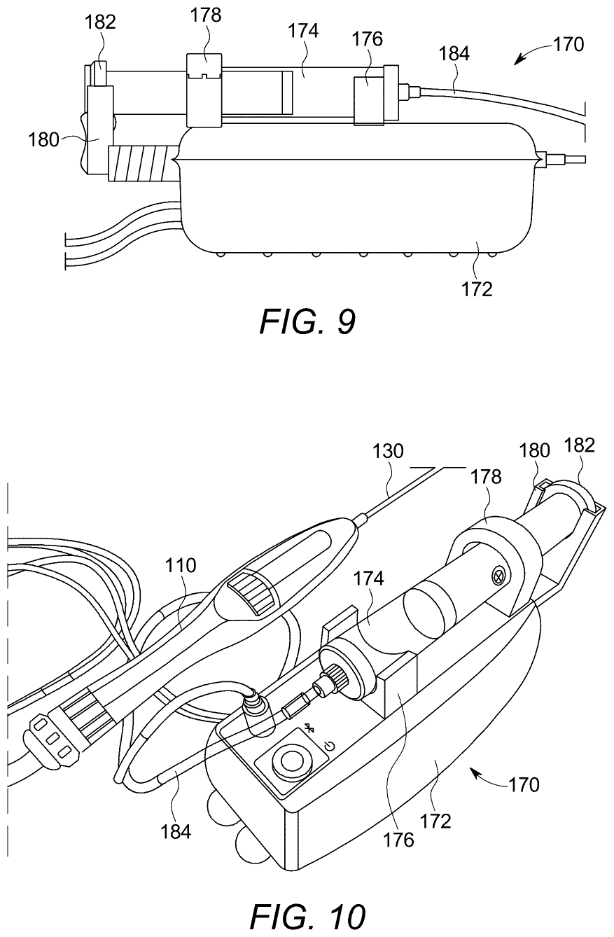

In some examples, the delivery catheter 130 is steerable. For example, one or more steering wires may extend through a wall of the delivery catheter 130, with one end of the steering wire coupled to a steering ring coupled to the delivery catheter 130, and another end of the steering wire operable coupled to a steering actuator on the handle 110. In such examples, as the steering actuator is actuated, the steering wire is tensioned or relaxed to cause deflection or straightening of the delivery catheter 130 to assist with steering the delivery catheter 130 to the desired position within the patient. For example, FIG. 5 is an enlarged view of the handle 110. Handle 110 may include a steering knob 112 that, upon rotation, tensions or relaxes the steering wires to deflect the distal end of the delivery catheter 130. Handle 110 may include a slot 118 with an indicator extending therethrough, the indicator moving along the slot 118 as the delivery catheter 130 deflects (e.g., the indicator moves proximally as deflection increases). If included, the indicator and slot 118 may provide the user an easy reference of how much the delivery catheter 130 is deflected at any given point. However, it should be understood that the steering functionality may be omitted in some examples, and in other examples steering actuators other than knobs may be utilized. Further, in some examples, including those shown in FIGS. 6 to 7, the delivery catheter 130 includes an outer catheter 132, and an inner catheter 134. The inner catheter 134 may also be referred to as a guidewire catheter. The steering functionality may be provided in either the outer catheter 132, or the inner catheter 134, or in both catheters. However, in some examples, a separate steering catheter 135 may be provided. For example, as shown in FIG. 4, the steering catheter 135 may be positioned outside of the outer catheter 132 and may terminate just proximal to the balloon 136. With this configuration, deflection of the steering catheter 135 will also cause deflection of the outer catheter 132 and the inner catheter 134 which are both nested within the steering catheter 135.

Still referring to FIGS. 4 to 5, the delivery system 100 may include additional functionality to assist with positioning the prosthetic heart valve 10. For example, in the illustrated example, handle 110 includes a commissure alignment actuator 114, which may be positioned near a proximal end of the handle or at any other desired location. In the illustrated example, the commissure alignment actuator 114 is in the form of a rotatable knob, although other forms may be suitable. The commissure alignment knob 114 may be rotationally coupled to a portion of the delivery catheter 130 supporting the prosthetic heart valve 10. For example, the commissure alignment actuator 114 may be rotationally coupled to an inner catheter 134 which supports the prosthetic heart valve 10 in the crimped condition. With this configuration, rotating the commissure alignment knob 114 may cause the inner catheter 134 to rotate about its longitudinal axis, and thus cause the prosthetic heart valve 10 to rotate about its longitudinal axis. If a commissure alignment actuator 114 is included, it may be used to help ensure that, upon deployment of the prosthetic heart valve 10 into the native valve annulus, the commissures of the prosthetic heart valve are in rotational alignment with respective ones of the native valve commissures (e.g. within +/−2.5 degrees of rotational alignment, within +/−5 degrees of rotational alignment, within +/−10 degrees of rotational alignment, within +/−15 degrees of rotational alignment, etc.). Although commissure alignment actuator 114 is shown in this example as a knob positioned at or near a proximal end of the handle 110, it should be understood that the actuator 114 may take forms other than a knob, may be positioned at other suitable locations, and may be omitted entirely if desired.

Still referring to FIGS. 4 to 5, the delivery system 100 may include even further functionality to assist with positioning the prosthetic heart valve 10. For example, in the illustrated example, handle 110 includes an axial alignment actuator 116, which may be positioned near a proximal end of the handle, including distal to the commissure alignment actuator 114, or at any other desired location. In the illustrated example, the axial alignment actuator 116 is in the form of a rotatable knob, although other forms may be suitable. The axial alignment knob 116 may be operably coupled to a portion of the delivery catheter 130 supporting the prosthetic heart valve 10. For example, the axial alignment actuator 116 may include internal threads that engage external threads of a carriage that is coupled to the inner catheter 134 which supports the prosthetic heart valve 10 in the crimped condition. In such an example, the carriage may be rotatably fixed to the handle 110. With this configuration, rotating the axial alignment knob 116 may cause the carriage to advance distally or retract proximally as the inner threads of the axial alignment knob 116 mesh with the external threads of the carriage, but the carriage is prevented from rotating. As the carriage advances distally or retracts proximally, the inner catheter 134 may correspondingly advance distally or retract proximally, and thus cause the prosthetic heart valve 10 to advanced distally or retract proximally. It should be understood that, if axial alignment actuator 116 is included, it may have a small total range of motion. In other words, the rough or coarse axial alignment between the prosthetic heart valve 10 and native valve annulus may be achieved by physically advancing the entire delivery catheter 130 by pushing it through the vasculature while holding the handle 110. However, for fine and more controlled adjustment of the axial position of the prosthetic heart valve 10 relative to the native valve annulus, which may be performed just prior to or during deployment of the prosthetic heart valve 10, the axial alignment knob 116 may be used. If an axial alignment actuator 116 is included, it may be used to help ensure that, upon deployment of the prosthetic heart valve 10 into the native valve annulus, the inflow end of the of the prosthetic heart valve is in axial alignment with the inflow aspect of the native valve annulus (e.g. within +/−0.5 mm of axial alignment, within +/−1.0 mm of axial alignment, within +/−1.5 mm of axial alignment, within +/−2.0 mm of axial alignment, etc.). Although axial alignment actuator 116 is shown in this example as a knob positioned at or near a proximal end of the handle 110, it should be understood that the actuator 116 may take forms other than a knob, may be positioned at other suitable locations, and may be omitted entirely if desired.

In addition to steering and positioning actuators, delivery system 100 may include a balloon actuator 120. In the illustrated example, balloon actuator 120 is positioned on the handle 110 near a distal end thereof, and is provided in the form of a switch. Balloon actuator 120 may be actuated to cause inflation or deflation of a balloon 136 that is part of the delivery system 100. For example, referring briefly to FIGS. 6 to 7, the delivery system 100 may include a balloon 136 (a delivery balloon) that overlies a distal end of inner catheter 134 and which receives the prosthetic heart valve 10 in a crimped condition thereon. In the example illustrated in FIG. 6, the balloon 136 includes a proximal pillowed portion 136a, a distal pillowed portion 136b, and a central portion over which the prosthetic heart valve 10 is crimped. The proximal pillow 136a and the distal pillow 136b may form shoulders on each side of the prosthetic heart valve 10, which may help ensure the prosthetic heart valve 10 does not move axially relative to the balloon 136 and/or inner catheter 134 during delivery. The shoulder formed by the distal pillow 136 may also help protect the inflow edge of the prosthetic heart valve 10 from contact with the anatomy during delivery. For example, during a transfemoral delivery, as the distal end of the delivery catheter 130 traverse the sharp bends of the aortic arch (or during initial introduction into the patient), there is a relatively high likelihood the inflow end of the prosthetic heart valve 10 (which is the leading edge during transfemoral delivery) will contact a vessel wall (or a components of an introduction system) causing dislodgment of the prosthetic heart valve 10 relative to the balloon 136. The distal pillow 136 may tend to have an equal or larger outer diameter than the inflow end of the prosthetic heart valve 10 (when the prosthetic heart valve 10 is crimped and the balloon 136 is deflated), which may help ensure the inflow edge of the prosthetic heart valve 10 does not inadvertently contact another structure during delivery. In some examples, the pillowed portions 136a, 136b may be formed via heat setting. Additional related features for use in similar balloon catheter delivery systems are described in greater detail in U.S. Patent Application Publication No. 2024/0148501, filed Oct. 31, 2023 and titled “Prosthetic Heart Valve Delivery and Trackability,” the disclosure of which is hereby incorporated by reference herein.

In order to deploy the prosthetic heart valve 10, the balloon 136 is inflated, for example by actuating the balloon actuator 120 to force fluid (such as saline, although other fluids, including liquids or gases, could be used) into the balloon 136 to cause it to expand, causing the prosthetic heart valve 10 to expand in the process. For example, the balloon actuator 120 may be pressed forward or distally to cause fluid to travel through an inflation lumen within delivery catheter 130 to inflate the balloon 136. FIG. 7 illustrates an example of the balloon 136 after being inflated, with the prosthetic heart valve 10 omitted from the figure for clarity. In the illustrated example, the balloon 136 may be formed to have a distal end that is fixed to a portion of an atraumatic distal tip 138. The distal tip 138 may be tapered to help the delivery catheter 130 move through the patient's vasculature more smoothly. A proximal end of the balloon 136 may be fixed to a distal end of outer catheter 132. The inflation lumen may be the space between the outer catheter 132 and the inner catheter 134, or in other embodiments may be provided in a wall of the inner catheter 134, or in any other location that fluidly connects the interior of the balloon 136 to a fluid source outside of the patient that is operable coupled to the delivery system 100.

Referring to FIG. 7, in some examples, a mounting shaft 140 may be provided on the inner catheter 134. A proximal stop 142 and/or a distal stop 144 may be provided, for example at opposite ends of the mounting shaft 140. If the mounting shaft 140 is included, it may provide a location on which the prosthetic heart valve 10 may be crimped. If the proximal stop 142 and/or distal stop 144 is provided, they may provide physical barriers to the prosthetic heart valve 10 moving axially relative to the balloon 136. In one example, the proximal stop 142 may taper from a larger distal diameter to a smaller proximal diameter, and the distal stop may taper from a larger proximal diameter to a smaller distal diameter. The spacing between the proximal stop 142 and the distal stop 144, if both are included, may be slightly larger than the length of the prosthetic heart valve 10 when it is crimped over mounting shaft 140. However, it should be understood that one or both of the stops 142, 144 may be omitted, and the mounting shaft 140 may also be omitted. If the mounting shaft 140 is included, it is preferably axially and rotationally fixed to the inner catheter 134 so that movement of the inner catheter 134 causes corresponding movement of the mounting member 140, and thus the prosthetic heart valve 10 when mounted thereon.

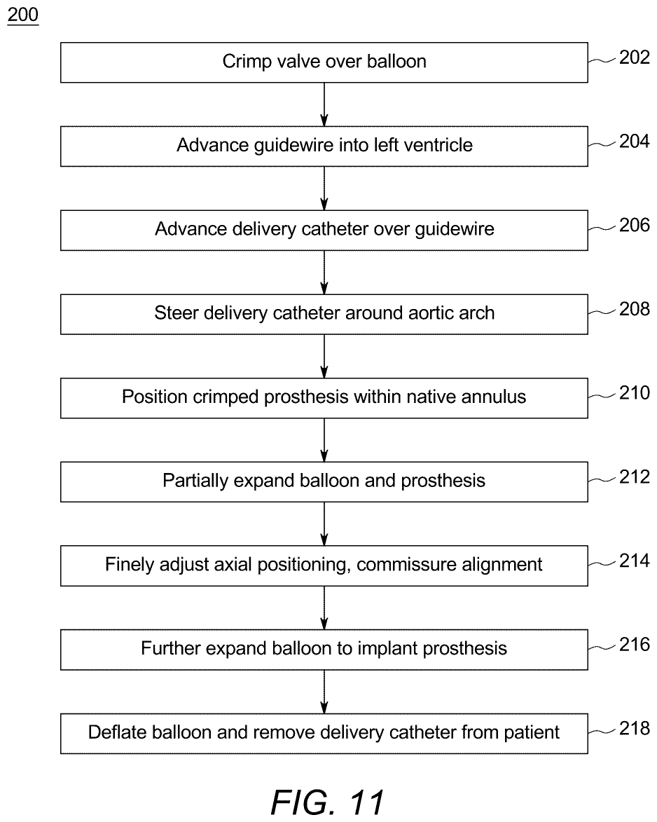

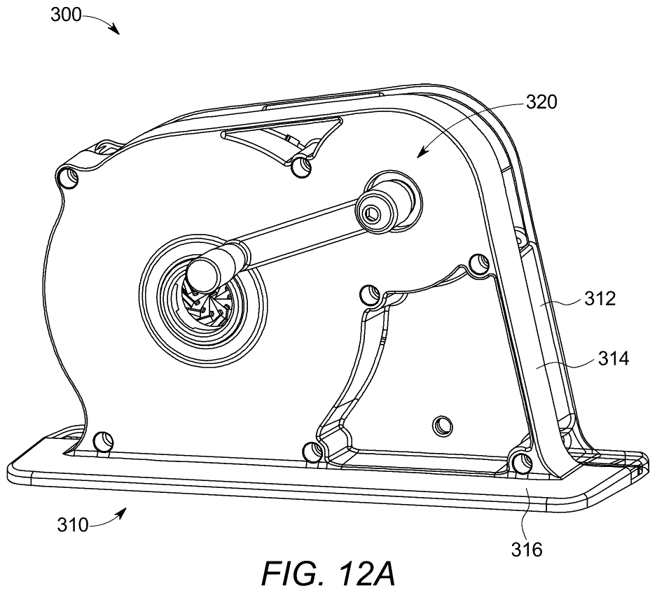

Before describing the use of balloon actuator 120 in more detail, it should be understood that in some embodiments, the balloon actuator 120 may be omitted and instead a manual device, such as a manual syringe, may be provided along with delivery system 100 in order to manually push fluid into balloon 136 during deployment of the prosthetic heart valve 10. However, in the illustrated example of delivery system 100, the balloon actuator 120 provides for a motorized and/or automated (or semi-automated) balloon inflation functionality. For example, FIG. 8 and FIG. 9 illustrate an example of a balloon inflation system 170. Balloon inflation system 170 may include a housing 172 that houses one or more components, which may include a motor, one or more batteries, electronics for control and/or communication with other components, etc. Housing 172 may include one or more fixed cradles to receive a syringe 174. In the illustrated embodiment, a distal cradle 176 is provide with an open “C”- or “U”-shaped configuration so that the distal end of the syringe 174 may be snapped into or out of the distal cradle 176. A proximal cradle 178 may also be provided, which may have a “C”- or “U”-shaped bottom portion hingedly connected to a “C”- or “U”-shaped top portion. This configuration may allow for the proximal end of the outer body of the syringe 174 to be snapped into the bottom portion of proximal cradle 178, and the top portion of proximal cradle 178 may be closed and connected to the bottom portion to fully circumscribe the outer body of the syringe 174 to lock the syringe 174 to the housing 172. It should be understood that more or fewer cradles, of similar or different designs, may be included with housing 172 to help secure the syringe 174 to the housing 172 in any suitable fashion.

The balloon inflation system 170 may include a moving member 180. In the illustrated embodiment, moving member 180 includes a “C”- or “U”-shaped cradle to receive a plunger handle 182 of the syringe 174 therein, the cradle being attached to a carriage that extends at least partially into the housing 172. The carriage of the moving member 180 may be generally cylindrical, and may include internal threading that mates with external threading of a screw mechanism (not shown) within the housing 172 that is operably coupled to a motor. In some embodiments, the carriage may have the general shape of a “U”-beam with the flat face oriented toward the top. The moving member 180 may be rotationally fixed to the housing 172 via any desirable mechanism, so that upon rotation of the screw mechanism by the motor, the moving member 180 advances farther into the housing 172, or retracts farther away from the housing 172, depending on the direction of rotation of the screw mechanism. While the plunger handle 182 is coupled to the moving member 180, advancement of the moving member 180 forces fluid from the syringe 174 toward the balloon 136, while retraction of the moving member 180 withdraws fluid from the balloon 136 toward the syringe 174. It should be understood that the motor, or other driving mechanism, may be located in or outside the housing 172, and any other suitable mechanism may be used to operably couple the motor or other driving mechanism to the moving member 180 to allow for axial driving of the plunger handle 182.

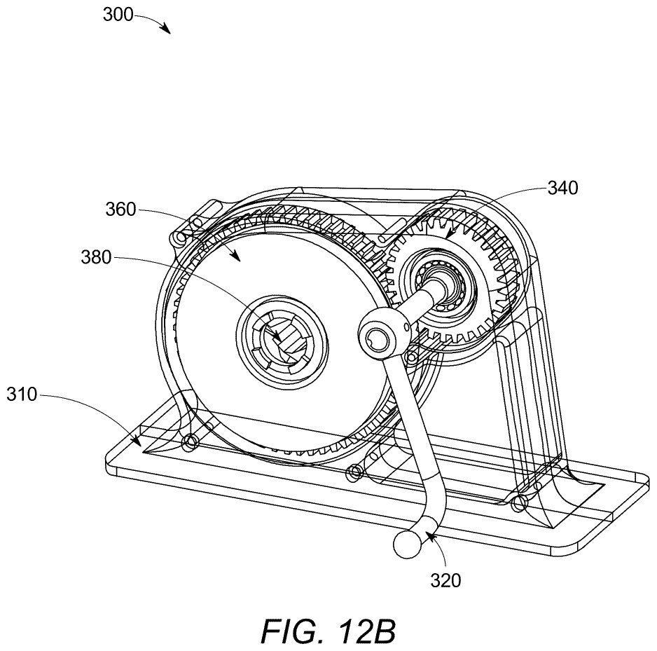

As shown in each of FIG. 8, FIG. 9, and FIG. 10, the distal end of syringe 174 may be coupled to tubing 184 that is in fluid communication with an inflation lumen of delivery catheter 130 that leads to the balloon 136 at or near the distal end of the delivery system 100. Tubing 184 may allow for the passage of the fluid (e.g., saline) from the syringe 174 toward the balloon 136, or for withdrawal of fluid from the balloon 136 toward the syringe 174, for example based on whether the balloon actuator 120 is pressed forward or backward.

Although not separately numbered in FIG. 8, FIG. 9, and FIG. 10, the housing 172 may include one or more cables extending from the housing, for example to allow for transmission of power (e.g., from AC mains or another component with which the cable is coupled) and/or transmission of data, information, control commands, etc. For example, one cable may couple the housing 172 to handle 110 so that controls on the handle 110 (e.g., balloon actuator 120) may be used to activate the balloon inflation system 170 in the desired fashion. Another cable may couple to a computer display or similar device to provide information regarding the inflation of the balloon 136. However, it should be understood that any transmission of data or information may be provided wirelessly instead of via a wired connection, for example via a Bluetooth or other suitable connection. Additional and related features of balloon inflation system 170, related systems, and the uses thereof are described in U.S. patent application Ser. No. 18/311,458, the disclosure of which is hereby incorporated by reference herein.

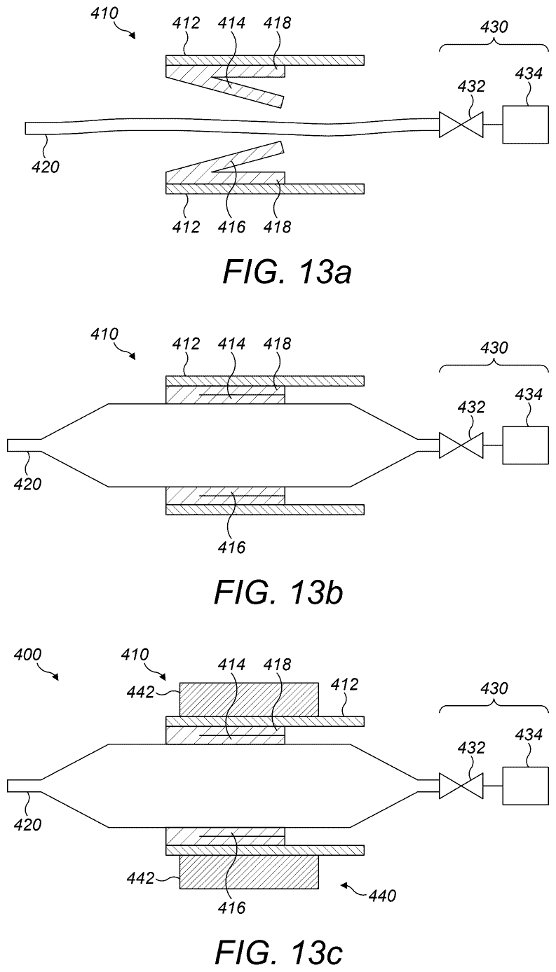

FIG. 11 is a flowchart showing exemplary steps in an implantation procedure 200 to implant the prosthetic heart valve 10 of FIG. 1 into a patient using the delivery system 100 of FIG. 4. However, it should be understood that not all of the steps shown in connection with implantation procedure 200 need to be performed, and various steps not explicitly shown and described in connection with procedure 200 may be performed as part of the implantation procedure. At the beginning of the procedure 200 in step 202, the prosthetic heart valve 10 may be collapsed over or crimped onto balloon 136, with the balloon 136 being mostly or entirely deflated after the crimping procedure. It should be understood that crimping step 202 may be performed at any time prior to the procedure, including at the beginning of the procedure, or at an earlier stage before the delivery system 100 is provided to the end user. In other words, the crimping step 202 may be performed during a manufacturing stage of the delivery system 100 and/or prosthetic heart valve 10. During an early stage of the implantation procedure 200, a guidewire GW may be advanced into the patient in step 204, for example via the femoral artery, around the aortic arch, through the native aortic valve, and into the left ventricle. The guidewire GW may be used as a rail for other devices that need to access this pathway. For example, in step 206, the atraumatic distal tip 138 may be advanced over the proximal end of the guidewire GW, and the delivery catheter 130 may be advanced over guidewire GW toward the native aortic valve. During this initial advancement of the delivery catheter 130 into the patient, the introducer 150 (if included) may be positioned distally, for example so that it covers the prosthetic heart valve 10 or so that it is positioned just proximal to the prosthetic heart valve 10. Advancement of the delivery catheter 130 and introducer 150 may continue until a proximal hub of the introducer is in contact with the patient's skin (or in contact with another device that enters the patient's femoral artery. At this point, the introducer 150 may stop moving axially relative to the patient, with the delivery catheter 130 continuing to advance relative to the introducer 150. If steering capability is provided, the delivery catheter 130 may be steered or deflected at any point to assist with achieving the desired pathway of the delivery catheter 130. As on example, in step 208, the steering knob 112 may be actuated to deflect the distal end of the delivery catheter 130 as it traverses the sharp bends of the aortic arch. Advancement of the delivery catheter 130 may continue in step 210 until the prosthetic heart valve 10, while still crimped or collapsed, is positioned within the native aortic valve annulus. With the desired position achieved, the balloon 136 may be partially inflated, for example by pressing balloon actuator 120 forward, to partially expand the prosthetic heart valve 10 in step 212. In some examples, it is desirable to expand the prosthetic heart valve 10 only partially in step 212, because the position of the prosthetic heart valve 10 (including rotational and/or axial positioning) relative to the native aortic valve annulus may shift during this partial expansion. After the partial expansion of step 212, the user may examine the positioning of the prosthetic heart valve 10 relative to the native aortic valve annulus. If desired, in step 214, the axial positioning of the partially-expanded prosthetic heart valve 10 relative to the native aortic valve annulus may be finely adjusted (e.g., by actuating axial alignment actuator 116) and/or the rotational orientation of the prosthetic heart valve 10 relative to the native aortic valve may be finely adjust (e.g., by actuating commissure alignment actuator 114). When the desired axial alignment is achieved and the desired rotational alignment (e.g., rotational alignment between the prosthetic commissure and the native commissures) is achieved, the balloon 136 may be fully expanded in step 216 to fully expand the prosthetic heart valve 10 and to anchor the prosthetic heart valve 10 in the native aortic valve annulus in the desired position and orientation. After deployment is complete, the balloon 136 may be deflated in step 218, for example by pressing actuating balloon 120 backward, and the delivery catheter 130 and guidewire GW may be removed from the patient to complete the procedure. It should be understood that the nine steps shown in FIG. 11 as part of procedure 200 are merely exemplary of a single example of an implantation procedure, and steps shown may be omitted, steps not shown may be included, and steps may be provided in any order deemed appropriate by the physician and/or medical personnel. In one example, the delivery catheter 130 may be guided to the right atrium and/or right ventricle for a tricuspid valve or pulmonary valve procedure. In another example, the delivery catheter 130 may be guided to the left atrium and/or left ventricle for a mitral valve procedure.

Although various components of a prosthetic heart valve 10 and delivery system 100 are described above, it should be understood that these components are merely intended to provide better context to the systems, features, and/or methods described below. Thus, various components of the systems described above may be modified or omitted as appropriate without affecting the systems, features, and/or methods described below. For example, prosthetic heart valves other than the specific configuration shown and described in connection with FIGS. 1 to 3 may be used with delivery systems other than the specific configuration shown and described in connection with FIGS. 4 to 10 as part of an implantation procedure that uses steps other than the specific configuration shown and described in connection with FIG. 11, without affecting the inventive systems, features, and/or methods described below.

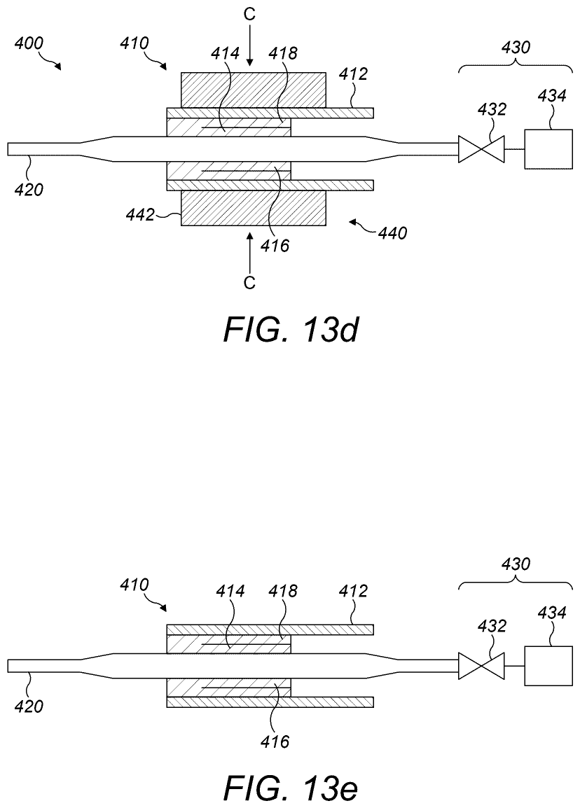

One example of a crimping device 300 (also referred to as a “crimper”) is shown in FIG. 12A. FIG. 12B shows the crimping device 300 with a casement or housing assembly 310 thereof in partial phantom. As shown in FIGS. 12A and 12B, the crimping device 300 may generally include a housing assembly 310, an actuator or handle assembly 320, a pinion or first gear assembly 340, a large or second gear assembly 360, and an iris assembly 380. It should be understood that, although the word “assembly” is used above, an “assembly” in some embodiments may include either a single component or multiple components that are structurally or functionally related. On a general level, the crimper 300 may be used by placing a device (such as a prosthetic heart valve positioned on a support member such as a balloon, compressible member or another component of a delivery device, or such as a stent) into the iris assembly 380 while the device is in a relatively expanded condition and while the iris assembly 380 is in a relatively opened condition. Then, the user may rotate the handle assembly 320 to cause the pinion assembly 340 to rotate, which in turn causes the large gear assembly 360 to rotate, which in turn forces the iris assembly 380 to transition to a relatively closed state, forcibly collapsing the device therein into a relatively collapsed condition. Additional related features of crimpers using in the preparation of similar balloon catheter delivery systems are described in greater detail in U.S. Provisional Patent Application No. 63/697,858, filed Sep. 23, 2024 and titled “Mechanical Crimper” the disclosure of which is hereby incorporated by reference herein, the crimpers of which may be used in any of the methods discussed herein. Further crimpers known in the art may also be used in the methods discussed herein and to crimp or collapse the prosthetic heart valves discussed herein.

Improvements are sought to improve the preparation of the prosthetic heart valves 10. For example, during delivery and after crimping, the valve 10 must have a small delivery diameter (crimp profile). Furthermore, the properties of the leaflets 90 within the valve and the lifespan and performance of the valve 10 are particularly important.

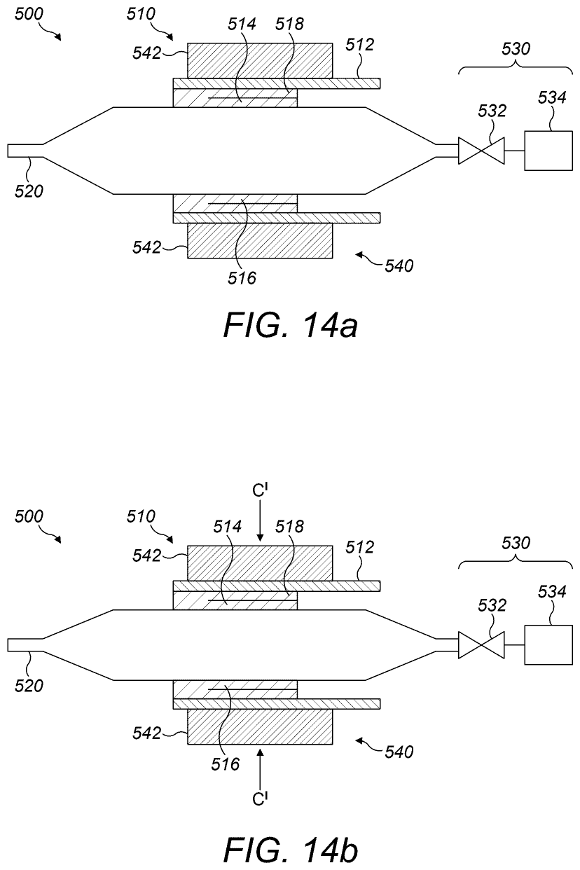

Various methods and systems for preparing prosthetic heart valves are presented below with reference to FIGS. 13 to 20. These methods and systems use support members such as balloons and compressible bodies (e.g. formed of rubber or foam) to flatten or push the leaflets of a valve towards the surrounding stent and to help prevent bunching or folding of these leaflets during crimping. These methods and systems may be utilised individually or in combination to improve the size of the crimped valve during delivery and lifespan and performance of valves after deployment.

The methods and systems presented below may be used during the preparation of any of the valves 10 and delivery devices 100 previously discussed with reference to FIGS. 1 to 10, and may be performed using the crimpers of FIG. 12. For instance, the methods discussed below may be performed as part of crimping step 202 shown in FIG. 11. The components described in relation to these drawings may include any of the features, materials and advantages described above with reference to the previous embodiments. The schematic cross sections within these figures are simplified to aid understanding. Notably, various features of delivery systems 100 discussed above are omitted for clarity in these drawings. For instance, the balloons discussed below may be coupled to or mounted on the catheters and actuators discussed above and/or attached to these catheters and actuators after the methods described below are complete.

FIGS. 13a to 13e show in schematic cross section, sequential steps of an exemplary method of preparing an expandable valve for delivery. The figures additionally illustrate a system 400 configured to perform this method.

As shown in FIG. 13a, there is initially provided or received a balloon expandable prosthetic heart valve 410 in an expanded state. The balloon expandable valve 410 comprises a collapsible stent 412, two opposing leaflets 414, 416 (although valves with three or more leaflets may also be used), and an optional inner cuff 418 separating the stent 412 from the leaflets 414, 416. The stent 412 is tubular and may comprise a cellular structure as discussed above. The prosthetic heart valve 410 may comprise further cuffs or skirts arranged inside or outside the stent 412.

As is also seen from FIG. 13a, in a first step of the method an inflatable balloon 420 (a support body) is inserted into the expanded valve 410 whilst the balloon 420 is in a deflated state. The balloon 420 is positioned within the stent 412 and extends through the aperture or lumen defined between the leaflets 414, 416. Herein, by “deflated” (also referred to as uninflated) it is understood that an internal pressure within the balloon is at or below atmospheric pressure. As such, the balloon's surface will be slack and easily deformed. In further examples of the method, the balloon 420 may be partially inflated when inserted into the expanded valve 410. By “partially inflated” it is understood that the balloon 420 is inflated such that its internal pressure remains relatively close to atmospheric pressure and where its external dimensions have not changed significantly relative to its uninflated form. In each case the external dimensions of the deflated or partially inflated balloon 420 are preferably less than the internal diameter of the stent 412 and preferably less than the distance between the leaflets 414, 416 when the valve 410 is in its expanded form. For instance, during insertion into the prosthetic heart valve, the maximum cross-sectional dimension of the balloon may be less than 75% of the internal diameter of the stent 412, and more preferably less than 50% of the internal diameter of the stent 412, when the valve is in its expanded form.

The balloon 420 is connected to a pressure regulation system 430. The pressure regulation system 430 is configured to inflate the balloon 420 and control the pressure within the balloon 420. The pressure regulation system 430 comprises a fluid supply device 434 (e.g. a pump or pressurised fluid reservoir) and a pressure regulator 432 (e.g. a valve, such as a pressure release valve). Alternative arrangements of the pressure regulation system 430 are also possible, such as a syringe with a pressure gauge. Similarly, in further examples the balloon 420 may be connected to a fluid supply device 434 without a pressure regulator. The fluid supply device 434 may be configured to supply a liquid such as saline, a gas such as air or nitrogen or any other suitable fluid to inflate the balloon. In some examples the pressure regulation system 430 and/or fluid supply device 434 may be the balloon actuators 110 and balloon inflation systems 170 discussed above with reference to FIGS. 4 to 10. Alternatively, the balloon may be disconnected from the pressure regulation system 430 and/or fluid supply device 434 after the valve 410 has crimped.

After insertion into the expanded prosthetic heart valve 410, the balloon 420 is inflated to a crimping pressure such that the balloon 420 contacts the leaflets 414, 416 of the prosthetic heart valve 410 as shown in FIG. 13b. As such, the volume of fluid (e.g. saline or another liquid) within the balloon 420 may be increased such that the balloon expands to contact the leaflets 414. Additionally or alternatively, the internal pressure of the balloon 420 may optionally be increased above atmospheric pressure such that the balloon 420 expands. In its inflated form the balloon 420 may have an external diameter that is greater than 80% internal diameter of the stent 412 in its expanded state, and more commonly will have an external diameter that is greater than 90% or 95% of the internal diameter of the stent 412 in its expanded state. As will be seen from FIG. 13b, expanding the balloon in this way acts to flatten the leaflets 414, 416 against the cuff 418 (if present) and an internal surface of the stent 412. Thus, the leaflets 414, 416 are placed in a “fully-open” arrangement, being flattened against the stent 412 (and cuff 418 or any other intervening layer). The expansion of the balloon 420 prior to crimping helps arrange the leaflets 414, 416 in a uniform, consistent position relative to the other components of the stent 412.

The prosthetic heart valve 410 is then crimped as shown in FIGS. 13c and 13d. In FIG. 13c the prosthetic heart valve 410 and the balloon 420 within it are inserted into a crimper 440 such as the crimping device 300 shown in FIGS. 12a and 12b. The crimper 440 comprises a plurality of jaws 442 (such as found in the iris of the crimping device 300 of FIG. 12) and is configured to apply radial forces to the exterior of the expandable prosthetic heart valve 410 to collapse the valve 410.

As shown, the balloon 420 is inserted into the prosthetic heart valve 410, the balloon 420 inflated to contact the leaflets 414, 416 of the valve 410, and subsequently the valve 410 and inflated balloon 440 are loaded into the crimper 440. However, this is not essential, and these steps may be performed in substantially any order. For instance, the method may involve inserting a deflated or partially inflated balloon 420 into the prosthetic heart valve 410, inserting the valve 410 and balloon 420 assembly into the crimper 440, and subsequently inflating the balloon 420.

Once inserted in the crimper 440, the valve 410 and balloon 420 are received between the jaws 442 of the crimper 440 (e.g. within the iris assembly 380 of the crimping device 300). Thereafter, the crimper is operated to close its jaws 442 and crimp (collapse) the prosthetic heart valve, as shown by arrows C in FIG. 13d. During crimping the external diameter of the prosthetic heart valve 410 is reduced from its expanded state as shown. In turn the prosthetic heart valve 410 applies force to the exterior of the balloon 420, reducing its external diameter too. The prosthetic valve 410 may be crimped to a delivery diameter—i.e. the intended or predetermined delivery diameter for the valve 410.

Throughout crimping the balloon 420 is maintained in contact with the leaflets 414, 416 of the prosthetic heart valve 410. Thus, during the crimping process the internal pressure within the balloon 420 may be maintained at a sufficient level above atmospheric pressure that the balloon 420 remains in contact with the leaflets 414, 416. This contact may be observed and confirmed during crimping by looking longitudinally along the system to the interface between the prosthetic heart valve 410 and the balloon 420.

In preferred examples, the internal pressure within the balloon 420 is maintained at a predetermined value throughout crimping. As such, as the diameter of the balloon 420 is decreased, fluid may be gradually released from the balloon 420 by the pressure regulation system 430 to maintain a constant internal pressure within the balloon. However, this is not essential and in further examples where the ballon is inflated using a compressible fluid (e.g. air, nitrogen or another gas) the fluid opening to the balloon 420 may be closed throughout crimping such that the pressure within the balloon 420 gradually increases as the balloon 420 is compressed. Equally, in further examples, the internal pressure within the balloon 420 may be gradually reduced as valve 410 is crimped. For instance, the internal pressure within the balloon 420 may be reduced from an initial value to atmospheric pressure during crimping, so that the balloon 420 becomes fully deflated as the valve 410 reaches its final diameter (its delivery diameter).

As the balloon 420 remains in contact with the leaflets 414, 416 during crimping as the diameter of the valve 410 is reduced, the balloon 420 supports the leaflets 414, 416 in position. Thus, the inflated balloon 420 received within the valve 410 applies a force or forces (e.g. frictional and/or radial forces) to the leaflets 414, 416, and these forces will restrict movement of the leaflets 414, 416, securing the leaflets in place. As such, the balloon 420 will restrict or act to prevent the leaflets 414, 416 of the valve 410 from moving relative to the stent 412 and other components of the valve 410. In particular, the balloon 420 will restrict the leaflets 414, 416 from moving circumferentially or longitudinally relative to the stent 412 and other components of the valve 410 as the prosthetic heart valve is crimped. This has the particular benefit that the leaflets 414, 416 are prevented from moving or bunching along the longitudinal axis of the stent 412. Indeed, in preferred examples the tissue of the leaflet is evenly distributed along the length of the valve, thereby allowing for a decrease in the maximum diameter of the valve.

Therefore, as shown in the figures, the leaflets 414, 416 may be maintained in a relatively consistent and uniform arrangement relative to the stent 412 and cuff 418 throughout crimping. This consistent and uniform arrangement of the leaflets 414, 416 enables the prosthetic heart valve 410 to be more consistently crimped to a smaller delivery diameter than possible without the use of the balloon 420 (support body). Moreover, supporting the leaflets 414, 416 throughout crimping avoids damage to the leaflets that can occur if leaflets are unconstrained during crimping.

In addition, since the leaflets 414, 416 remain in contact with the balloon 420 during crimping, they tend to be folded or interleaved with the material of the balloon around the circumference of the balloon 420. This interleaving, which is not shown in the figures for clarity, helps enable tight crimping of the valve 410. This interleaving may be observed if a crimped valve were cut in half and inspecting the arrangement of the leaflets 414, 416 and balloon 420.

Once the valve 410 is crimped, the assembly comprising the crimped prosthetic heart valve 410 and balloon 420 is removed from the crimper 440 as shown in FIG. 13e, the valve 410 being prepared for delivery. The balloon 420 may be deflated following the completion of the crimping process. Subsequently, the prosthetic heart valve 410 may be delivered and installed to a patient using the systems and methods discussed above. As such, the balloon 420 may be a delivery balloon and may be inserted with the prosthetic heart valve 410 crimped over it into a patient, positioned and inflated to expand and implant the prosthetic heart valve 410.

The balloon 420 may comprise a compliant balloon material such as polyurethane or silicone. Preferably the balloon has a single wall and has a wall thickness in the range from 0.025 mm to 0.15 mm, preferably from 0.05 mm to 0.1 mm (approximately 0.001 to 0.006 inch, preferably in the range from approximately 0.002 to 0.004 inches). However, other balloons are also possible. For instance, suitable balloons include both single walled balloons and balloons with multiple walls.

The balloon 420 is preferably configured to maintain a circular cross section throughout the crimping process. As such, the balloon 420 may maintain a circular cross section when inflated to a diameter at which it contacts the leaflets of the valve 410 when the valve 410 is in its expanded state (e.g. up to 50 mm dependent on the size of the valve 410) and at the end of crimping when the balloon 420 has an external diameter that corresponds to the internal diameter of the crimped valve 410 (e.g. less than 5 mm, dependent on the intended delivery diameter for the valve 410).

The pressure regulation system 430 (and optionally a further balloon actuation system used during delivery) are configured to inflate the balloon 420 by different amounts during crimping and delivery. The pressure applied to the balloon 420 during crimping must be sufficient to support the leaflets 414, 416 in position and restrict their movement relative to the other components of the valve 410, but not sufficient to expand the valve 410 or prevent the crimper from collapsing the valve 410. In contrast, during delivery, a greater internal pressure is provided to expand the balloon 420. The pressure applied during crimping may be less than 25% of the pressure applied during valve expansion, and is preferably less than 10%, more preferably less than 5%, of the pressure applied during valve expansion. For example, the internal pressure above atmospheric pressure applied to the balloon 420 during crimping—a crimping pressure—may be in the range from 0 to 203 kPa (approximately 0 to 2 atm and approximately 0 to 29.9 psi) and preferably in the range from 0 to 101 kPa (approximately 0 to 1 atm and approximately 0 to 14.6 psi). Whereas, the internal pressure above atmospheric pressure applied to the balloon 420 to expand the balloon during delivery—an expansion pressure—may be in the range from 507 kPa to 1.52 MPa (approximately 5 to 15 atm and approximately 73.5 to 218 psi) and is preferably in the range from 608 kPa to 1.01 MPa (approximately 6 to 10 atm and approximately 88.2 to 146 psi).

The pressure applied during crimping—the crimping pressure—may be predetermined and controlled by the pressure regulation system 430. Furthermore, this crimping pressure may be maintained at a constant pressure throughout crimping. Alternatively, the crimping pressure may be varied during the crimping process.

The method above is particularly suited for prosthetic heart valves having an external diameter in their full expanded state in the range from 15 to 50 mm, and preferably from 18 to 30 mm, and for crimping such valves to a final or delivery diameter in the range from 4 to 12 mm, preferably in the range from 5 to 10 mm. Thus, the external diameter of the prosthetic heart valve in its expanded state is preferably in the range from 2 to 5 times its delivery diameter.

A modified method for preparing the prosthetic heart valve 410 that may also be performed using the system 400 shown in FIGS. 13a to 13e involves a two-step crimping process.

In this modified method, a deflated or partially inflated balloon 420 is inserted into an expanded prosthetic heart valve 410, the balloon 420 is inflated such that it contacts the leaflets of the prosthetic heart valve 410 and the valve 410 and balloon 420 are inserted into the crimper as discussed above with reference to FIGS. 13a, 13b and 13c. Moreover, as previously mentioned, these steps may be performed in any order.

Thereafter, in a first crimping step, the prosthetic heart valve 410 is partially crimped to reduce its external diameter to an intermediate diameter that remains larger than the intended delivery diameter for the valve 410. During this step, the pressure within the balloon may be maintained at a sufficient level that the balloon 420 is maintained in contact with the leaflets 414, 416 throughout crimping. The balloon 420 applies frictional and radial forces to the leaflets 414, 416 to restrict their movement relative to the remaining components of the valve 410. The leaflets 414, 416 remain in a consistent, uniform arrangement during crimping. This first crimping step is analogous to the crimping step discussed above with reference to FIGS. 13a to 13e with the exception the valve 410 is not crimped to its delivery diameter and may involve any of the optional or preferable features of the crimping step discussed above.

Subsequently, the balloon 420 is deflated, and a second crimping step is performed. In this second crimping step the prosthetic heart valve 410 is crimped further to reduce its external diameter from the intermediate diameter—e.g. to its intended delivery diameter. Although the leaflets 414, 416 are not supported by the deflated balloon 420 during the second crimping step, they tend to remain in the relatively consistent, uniform arrangement encouraged by the balloon 420 in the first crimping step. As such, this two-step crimping process continues to offer improvements in the delivery size, performance and lifespan of the prosthetic heart valve 410.

Again, this modified method above is particularly suited for prosthetic heart valves having an external diameter in their full expanded state in the range from 15 to 50 mm, and preferably from 18 to 30 mm, and for crimping such valves to a final or delivery diameter in the range from 4 to 12 mm, preferably in the range from 5 to 10 mm. The intermediate diameter may be in the in the range from 8 to 20 mm, and is preferably in the range from 10 to 16 mm. The external diameter of the prosthetic heart valve in its expanded state may be in the range from 2 to 5 times the delivery diameter. Whilst the intermediate diameter may be in the range from 1.1 to 2 times the delivery diameter.

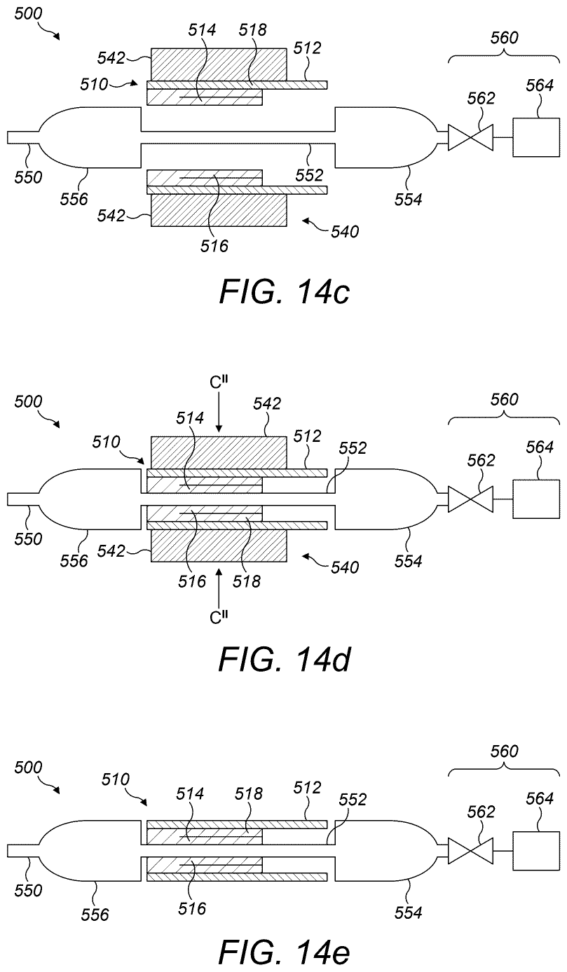

Sequential steps of a further method involving a two-step crimping process are shown in schematic cross section in FIGS. 14a to 14e. The method uses a similar system 500 to that shown in FIGS. 13a to 13e. Components of this system 500 which may comprise the same features and offer corresponding advantages to the equivalent components discussed above with reference to FIGS. 13a to 13e have had their reference signs incremented by 100.

In contrast to the approaches discussed above with reference to FIG. 13, the method of FIGS. 14a to 14e uses two balloons: a sacrificial balloon used to support the leaflets of a prosthetic heart valve during a first crimping step, and a delivery balloon onto which the valve is crimped further during a second crimping step, wherein the delivery balloon is configured to carry and expand the valve during delivery and implantation of the valve.

FIGS. 14a and 14b show a system 500 comprising an expandable prosthetic heart valve 510, a sacrificial balloon 520 and a connected first pressure regulation system 530, a delivery balloon 550 and an optional connected second pressure regulation system 560 and crimper 540. The expandable valve 510 comprises a collapsible stent 512, two opposing leaflets 514, 516 (although valves with three or more leaflets may also be used), and an optional inner cuff 518. The first pressure regulation system 530 is connected to the sacrificial balloon 520 and is configured to control its internal pressure during a first crimping step, comprising a pressure regulator 532 (e.g. a valve) and a fluid supply device 534 (e.g. a pump). The optional second pressure regulation system 560 is connected to the delivery balloon 550 and is configured to inflate the delivery balloon 550 to expand the valve 510 during implantation and is optionally configured to control the internal pressure of the delivery balloon 550 during the second crimping step. The second pressure regulation system 560 again comprises a pressure regulator 562 (e.g. a valve) and a fluid supply device 564 (e.g. a pump), although other arrangements are also possible. The crimper 540 comprises a plurality of jaws 542 and is configured to apply radial forces to collapse the expandable prosthetic heart valve 510.

The first and second pressure regulation systems 530, 560 may be separate and configured to individually regulate the pressure in the sacrificial and delivery balloons 510, 550, respectively. However, this is not essential and in further examples the first and second pressure systems may be the same system. For instance, the internal pressures within the sacrificial and delivery balloons 520, 550 may be regulated by the same pressure regulation system. This single pressure regulation system may be configured to connect to both balloons 520, 550 simultaneously, or alternately. In the latter example, the single pressure regulation system may be disconnected from the sacrificial balloon after a first crimping step and connected to the delivery balloon ahead of delivery.

In FIG. 14a the prosthetic heart valve 510 is arranged in an expanded state. The sacrificial balloon 520 is inserted into the prosthetic heart valve 510 and inflated to contact and apply forces to the leaflets 514, 516 of the valve 510. As shown, this flattens the leaflets 514, 516 against to the surrounding stent 512 and will restrict their movement. In addition, the valve 510 and balloon 520 are received between the jaws 542 of the crimper 540. The principles behind these steps are discussed above with reference to FIGS. 13a to 13c.

Thereafter, in a first crimping step shown in FIG. 14b, the crimper 540 is operated to close its jaws 542 and to crimp the valve 510. The movement of the jaws 542 during this step is shown by arrow C′ in the figure. During this first crimping step the external diameter (crimp profile) of the valve 510 is reduced from its expanded diameter to a support release diameter. Throughout this first crimping step the internal pressure within the sacrificial balloon 520 is maintained at a sufficient level such that the balloon remains in contact with the leaflets 514, 516 of the valve 510. Therefore, the sacrificial balloon 520 supports the leaflets 514, 516 throughout crimping, and applies force to the leaflets 514, 516 that restrict their movement relative to the remaining features of the valve 510 as the diameter of the valve 510 is reduced. The principles and benefits of this process are analogous to those discussed with reference to FIG. 13. Furthermore, similar pressures as discussed above with reference to FIG. 13 may be applied to the balloon during this crimping process.

Following the first crimping step the sacrificial balloon 520 may be fully or partially deflated and removed from within the prosthetic heart valve 510. Deflating the sacrificial balloon 520 will reduce the diameter of the balloon 520, causing it to release (separate) from the leaflets 514, 516. The sacrificial balloon 520 may then be removed from inside the valve 510 without affecting the positioning of the leaflets 514, 516.

Following the removal of the sacrificial balloon 520, a delivery balloon 550 is inserted into the partially crimped prosthetic heart valve 510, as shown in FIG. 14c. Thus, the delivery balloon 550 is inserted within the stent 512 and extends through the aperture defined between the leaflets 514, 516. The sacrificial balloon 520 may be removed from the valve 510 and/or the delivery balloon 550 inserted into the prosthetic heart valve 510 whilst the valve 510 remains in the crimper 540. However, this is not essential and the valve 510 may be removed from the crimper 540 for one or both of these steps.

The delivery balloon 550 is configured to carry the valve 510 during delivery and to expand the valve 510 to implant it into the patient. As previously discussed, the delivery balloon 550 is optionally connected to a second pressure regulation system 560 comprising a pressure regulator 562 and a fluid supply device 564. In further examples the delivery balloon 550 may be connected to an actuator or pressure regulation system after the prosthetic heart valve 510 has been prepared and shortly before delivery. The delivery balloon 550 comprises a central portion 552 over and onto which the prosthetic heart valve 510 is to be crimped, a proximal pillowed portion 554 positioned closer to the second pressure regulation system 560 than is the central portion 552, and a distal pillowed portion 556 positioned further from the second pressure regulation system 560 than is the central portion 552. The pillowed portions 554, 556 are configured to protect the edges of the prosthetic heart valve 510 during delivery, while also helping maintain the relative axial position between the valve 510 and the balloon 550 during delivery, as previously discussed in relation to FIGS. 6 and 7. As seen, the pillowed portions 554, 556 have a greater diameter than the central portion 552 of the delivery balloon 550 when the delivery balloon 550 is in a deflated state. The pillowed portions 554, 556 may comprise folded, pleated and/or twisted sections of the body of the delivery balloon 550. The folds, pleats and/or twists are configured to unfold or untwist as the delivery balloon 550 is inflated to expand the valve 510 during implantation. These folds, pleats and/or twists are typically formed or secured by heat treatment of the body of the balloon 550 prior to assembly.

Following insertion of the delivery balloon 550 into the partially crimped prosthetic heart valve 550, in a second crimping step shown in FIG. 14d the prosthetic heart valve 550 is crimped further from its support release diameter to a delivery diameter. During this second crimping step the crimper 540 is operated to close its jaws 542 further to crimp the valve 510 onto the central portion 552 of the delivery balloon 550. The leaflets 514, 516 of the prosthetic heart valve 510 are closed onto the outer surface of this central portion 552 of the balloon 550. The movement of the jaws 542 of the crimper 540 during this step is shown by arrow C″ in FIG. 14d.

Following the second crimping step, the assembly comprising the crimped prosthetic heart valve 510 and the delivery balloon 550 is removed from the crimper 540 as shown in FIG. 14e. As such, the delivery balloon 550 and prosthetic heart valve 510 may prepared for delivery. Thereafter the valve 510 may be delivered into and implanted into a patient using the delivery balloon as previously discussed.

The delivery balloon 550 is preferably inserted into the prosthetic heart valve 510 in a deflated state. In addition, the second crimping step is preferably performed whilst the delivery balloon 550 is in a deflated state. This offers the advantage that the pillowed portions 554, 556, which are formed before the delivery balloon 550 is inserted into the valve 510 and before the second crimping step, may be retained until delivery of the valve 510. Furthermore, although the leaflets 514, 516 of the valve 510 are not supported during the second crimping step, they tend to remain in the relatively consistent, uniform arrangement formed by the use of the sacrificial balloon 520 in the first crimping step. As such, this two-step crimping process continues to offer improvements in the delivery size, performance and lifespan of the prosthetic heart valve 510.

The method discussed with reference to FIGS. 14a to 14e is well suited for prosthetic heart valves having an external diameter in their full expanded state in the range from 15 to 50 mm, and preferably from 18 to 30 mm, and for crimping such valves to a final or delivery diameter in the range from 4 to 12 mm, preferably in the range from 5 to 10 mm. The support release diameter may be in the in the range from 8 to 20 mm, and is preferably in the range from 10 to 16 mm. The external diameter of the prosthetic heart valve in its expanded state may be in the range from 2 to 5 times the delivery diameter. Whilst the support release diameter may be in the range from 1.1 to 2 times the delivery diameter.

As discussed above, the delivery balloon 550 that comprises pre-formed pillowed portions 554, 556 is not inflated during the method shown in FIG. 14a to 14e. This maintains the pillowed portions 554, 556 in their folded, twisted or pleated shape. Typically inflating a balloon with pillowed portions will cause the folds, twists and/or pleats forming the pillowed portions to unfold as the balloon expands. These folds, twists and/or pleats, and the pillowed regions formed thereof, will not reform when the balloon is deflated.

In further examples, a balloon with pre-formed pillowed portions such as the delivery balloon 550 shown in FIGS. 14c to 14e may be inflated and used as a support body during crimping. To avoid the inflation of the balloon affecting the pre-formed pillowed portions, these pillowed portions may be constrained to prevent them unfolding during preparation of the valve assembly.

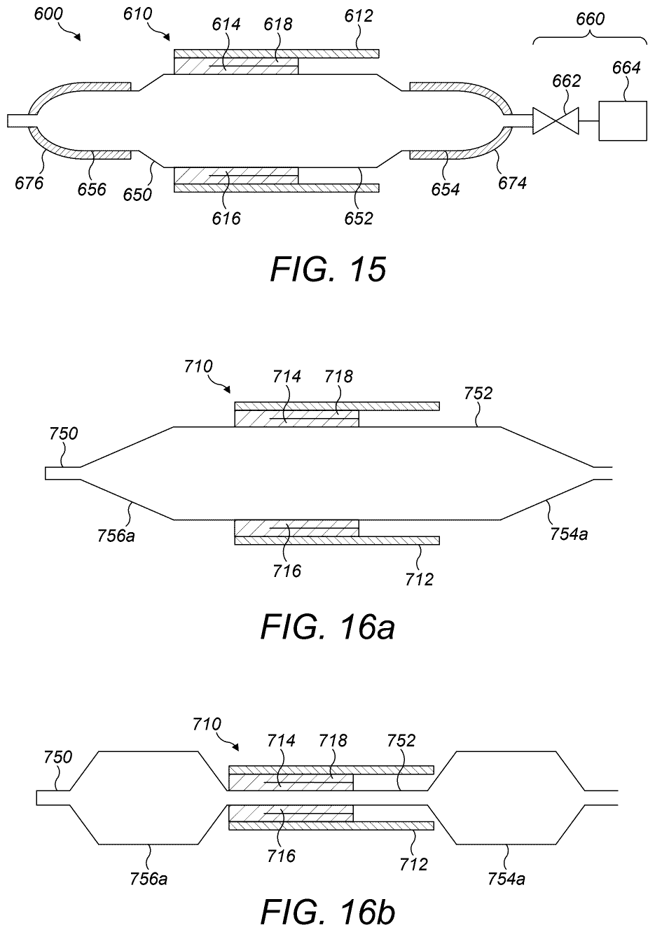

An example of system 600 configured to constrain the pillowed portions of a balloon is shown schematically in FIG. 15. Components of this system 600 which correspond to equivalent components of the system 500 discussed above with reference to FIGS. 14a to 14e and may share any of the features and advantages discussed above have had their reference signs incremented by a multiple of 100.

FIG. 15 shows in schematic cross section a system 600 comprising an expandable prosthetic heart valve 610, a balloon 650 (e.g. a delivery balloon), and a pressure regulation system 660. The prosthetic heart valve 610 comprises a collapsible stent 612, two opposing leaflets 614, 615 (although valves with three or more leaflets may also be used), and an optional inner cuff 618. The balloon 650 comprises a central portion 652 over and onto which the prosthetic heart valve 510 may be crimped, proximal pillowed portion 654 positioned closer to the second pressure regulation system 660 than is the central portion 652, and a distal pillowed portion 656 positioned further from the second pressure regulation system 660 than is the central portion 652. The pressure regulation system 660 is connected to the balloon 650 and is configured to control the internal pressure therein. The pressure regulation system 660 comprises a pressure regulator 662 and a fluid supply device 664, although other arrangements as also possible.

In addition, the system 600 shown in FIG. 15 comprises a pair of securing members in the form of two sheaths 674, 676. The sheaths 674, 676 are configured to prevent the expansion of the respective pillowed portions 654, 656 even if internal pressure is applied to the balloon 650. A proximal sheath 674 is configured to be arranged around the proximal pillowed portion 654, whilst a distal sheath 676 is configured to be arranged around the distal pillowed portion 656. The sheaths 674, 676 are rigid and are configured so that they may be slid onto each end of the balloon 650 and over the respective pillowed portions 654, 656. Each sheath 674, 676 comprises an interior surface which is configured to contact an exterior surface of the respective pillowed portion 654, 656. The rigid sheaths may be made of any suitable material for constraining pillowed regions including a rigid polymer, metal or metal alloy.

In FIG. 15, the prosthetic heart valve 610 is shown in an expanded state, with the balloon 650 inserted therethrough. The sheaths 674, 676 are arranged over the pillowed portions 654, 656 of the balloon 650. The balloon 650 is inflated so that its central portion 652 is expanded to contact the leaflets 614, 616 of the valve 610. As shown, the central portion 652 of the balloon pushes the leaflets 614, 616 of the valve 610 towards the stent 612, flattening them into a fully-open arrangement as discussed above with reference to FIGS. 13 to 14.

Unlike the central portion 652 of the balloon 650, the pillowed portions 654, 656 are not expanded, being constrained by the overlying sheaths 674, 676. Therefore, the sheaths 674, 676 allow the balloon 650 to be inflated within the prosthetic heart valve 610, and for the valve 610 to be crimped with internal support from the balloon 650 whilst the arrangement and shape of the pre-formed pillowed portions 654, 656 is retained.

In further examples, one or more of the sheaths may be replaced with an alternative securing member. For instance, a pillowed portion may be secured or constrained during inflation of a balloon and subsequent crimping using a band, an elastic or rubber band, a clamshell, a clamp, a cover or any other suitable device.

The assembly comprising the balloon 650, pressure regulation system 660 and sheaths 674, 676 may be used in place of the balloon 420 and pressure regulation system 430 in the methods of FIGS. 13a to 13e which involve a single crimping step. Equally, this assembly may be used in place of the delivery balloon and second pressure regulation system in the methods involving two crimping steps in FIGS. 14a to 14e. Furthermore, the assembly shown in FIG. 15 may be used in a modified version of the methods of FIGS. 14a to 14e, wherein the balloon 650 is used as a delivery balloon, and is inflated during the second crimping step to support the leaflets of the valve.

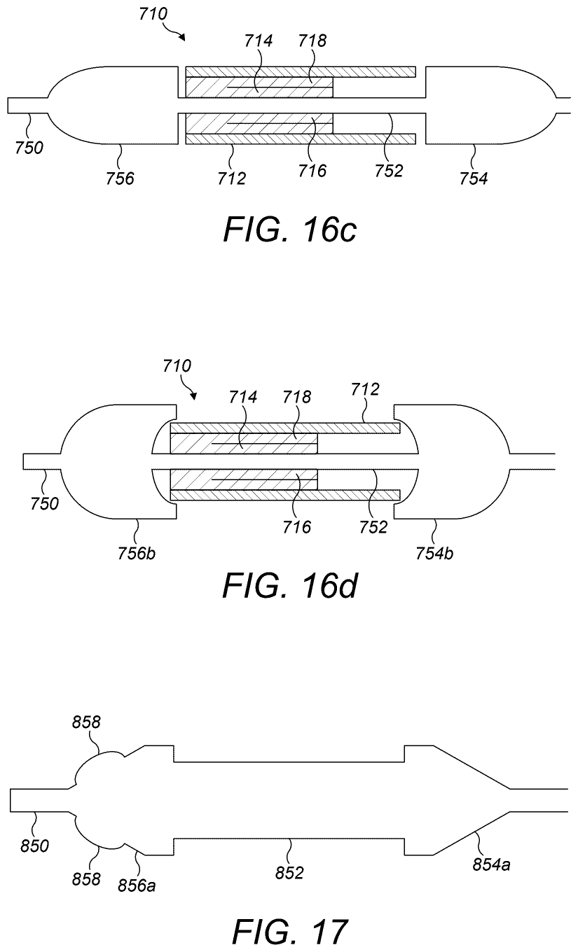

In still further examples, one or more structural features configured to protect a prosthetic heart valve may be formed into a balloon after the valve has been crimped onto said balloon. Examples of these balloons and methods for forming these protective structural features will be discussed with reference to FIGS. 16a to 16d. Components in these figures that correspond to components in FIGS. 13 to 15 have had their reference signs incremented by a multiple of 100. These components may share any of the features and advantages of the corresponding components discussed above.

FIG. 16a shows a prosthetic heart valve 710 and a balloon 750 in a similar arrangement to the ones seen in FIG. 13b. The prosthetic heart valve 710 is arranged in an expanded state, and comprises a stent 712, leaflets 714, 716 and an optional inner cuff 718. The balloon 750 is inserted through the prosthetic heart valve 710 and has been inflated such that a central portion 752 of the balloon 750 contacts and flattens the leaflets 714, 716 of the valve 710 as previously discussed. A pressure regulation system configured to control the internal pressure within the balloon as previously discussed may be connected to the balloon 750 but is omitted from these figures for clarity.

The balloon 750 is relatively long, having a proximal portion 754a that extends beyond the stent 712 in a proximal direction (right as shown) and a distal portion 756a that extends beyond the stent 712 in a distal direction (left as shown). Preferably the proximal portion 754a has a length that is at least 30% of the length of the stent, more preferably at least 50%, more preferably at least 75%. Additionally or alternatively, the distal portion 756a may have a length that is at least 30% of the length of the stent, more preferably at least 50%, more preferably at least 75%.

Following the insertion and inflation of the balloon 750, the prosthetic heart valve 710 may be crimped to form the arrangement shown in FIG. 16b. This crimping process may be performed using the same principles as discussed above with reference to FIGS. 13 to 15. As seen from FIG. 16b, the crimping process leaves the relatively large proximal and distal portions 754a, 756a of the balloon 750 free. After crimping the balloon 750 may be deflated, leaving the proximal and distal portions uninflated and slack.

The proximal and distal portions 754a, 756a may subsequently be folded, pleated and/or twisted to form a proximal pillowed portion 754 and a distal pillowed portion 756 respectively. This arrangement of a valve 710 crimped onto a balloon 750 with adjacent pillowed portions 754, 756 is shown in FIG. 16c. This assembly of a valve and balloon in which pillowed portions 756, 754 are formed after the crimping process is very similar to the assembly shown in FIG. 14e where pillowed portions were pre-formed.

Alternatively, after the balloon 750 is deflated, the proximal and distal portions 754a, 756a of the balloon 750 may be folded over the proximal and distal ends of the valve 710 respectively, as shown by proximal folded portion 754b and distal folded portion 756b in FIG. 16d. This folding of the balloon 750 over the ends of the valve 710 offers similar protection and stabilization to the valve 710 during delivery as the use of pillowed portions.

The pillowed portions and folded portions may be secured within the balloon using heat treatment. Increasing the temperature of the pillowed portions and folded portions may prevent these portions from unfolding or untwisting before the balloon 750 is inflated.

The balloons 750 shown in FIGS. 16c and 16d may be used in place of the balloon 420 shown in FIGS. 13a to 13e or the delivery balloon 550 shown in FIGS. 14a to 14e. Similarly, any of the other methods described herein of preparing prosthetic heart valves for delivery that involve a step of crimping the valve onto a delivery balloon may be modified to include a subsequent step in which pillowed portions and/or folded portions are formed into the respective balloon. It will be appreciated that balloons may be provided with one or more pillowed portions and/or one or more folded portions. As such, a balloon need not only comprise pillowed portions or folded portions.

In further examples, the balloon may be configured to assist in the creation of pillowed portions and/or folded portions (as shown in FIGS. 16c and 16d). For instance, the wall thickness of a balloon may be varied between the central portion of the balloon on which a valve will be crimped in use and the distal and/or proximal portions from which a pillowed or folded portion may be formed. Similarly, the cross-sectional dimensions of the balloon may be varied between the central portion and the distal portion and/or proximal portion.

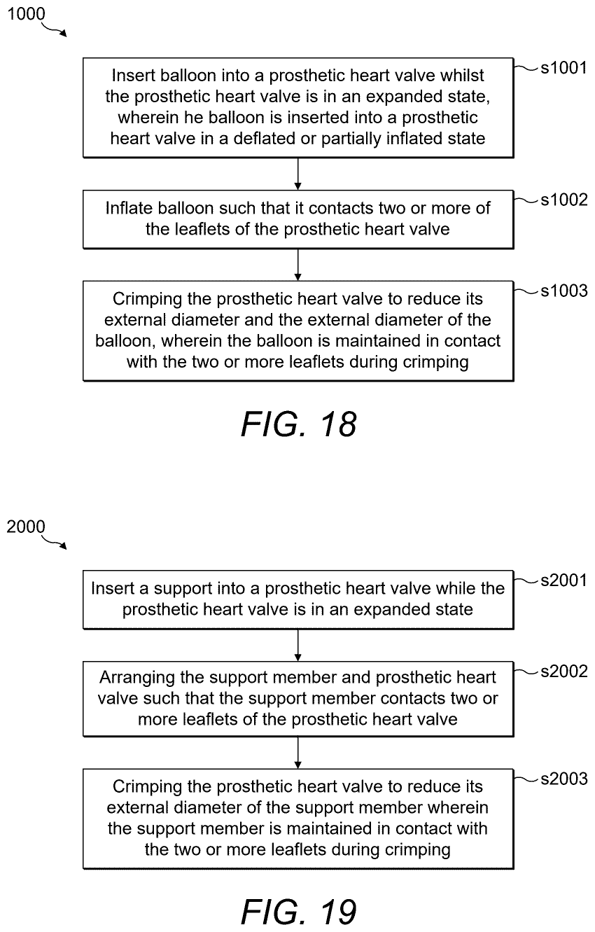

FIG. 17 shows a balloon 850 that comprises a central portion 852, a proximal portion 854a and a distal portion 856a. This balloon 850 may be used with any of the methods and systems discussed above. The central portion 852 of the balloon 850 is configured to receive a prosthetic heart valve in use, the valve being crimped over the central portion 852. The central portion 852 is recessed relative to the adjacent portions of the balloon, there being an increase in the cross-sectional dimension between the central portion 852 and the immediately adjoining parts of the proximal portion 854a and distal portion 856a.

Furthermore, the creation of pillowed or folded regions in the balloon 850 of FIG. 17 is simplified by providing additional material in the balloon in either the proximal portion or the distal portion. For example, in FIG. 17 the distal portion 856a of the balloon 850 comprises a projecting region 858 which has greater cross-sectional dimensions than a surrounding region of the balloon 850—e.g. the immediately distal region of the balloon 850 as shown. In otherwords, the material forming projecting region 858 extends beyond a plane defined by the surrounding portions of the distal portion of the balloon 840. The projection provides additional material to form into a pillow or to fold over the edge of a valve crimped onto the balloon 850. In further examples, a similar projecting region with increased cross-sectional dimensions when compared to the surrounding material may be formed in the proximal portion of a balloon.

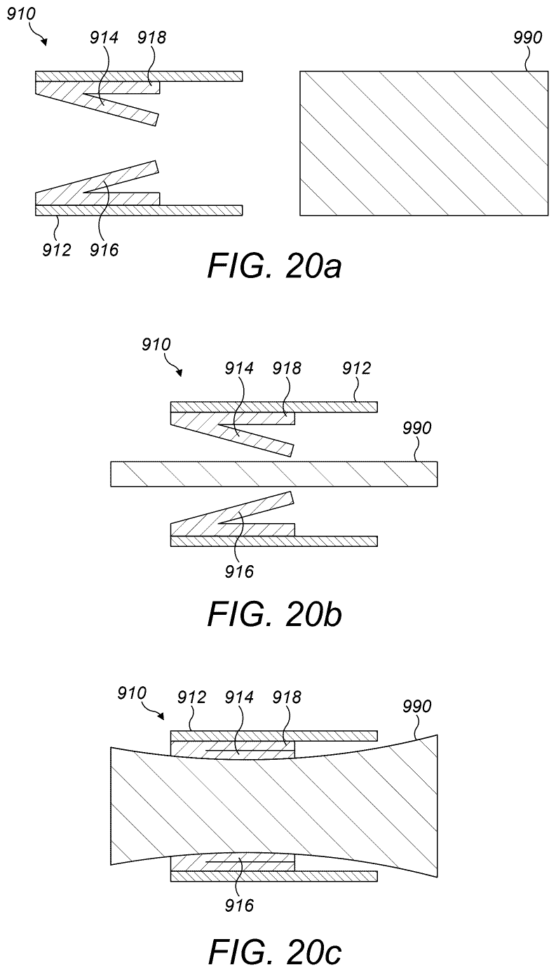

FIG. 18 shows a flow diagram of a method 1000 for preparing an expandable prosthetic heart valve for delivery, the steps of this method 1000 are common to all of the approaches discussed above that use a balloon as a support member and may be combined with any of the features discussed above.

In step s1001, a balloon (e.g. a sacrificial balloon, delivery balloon or other balloon) is inserted into an expandable prosthetic heart valve whilst the prosthetic heart valve is in an expanded state. The balloon is inserted into the prosthetic heart valve in a deflated or partially inflated state.

The prosthetic heart valve may comprise a stent and two or more leaflets arranged within. The balloon is preferably inserted such that it extends entirely through the stent and through the lumen (aperture) defined between the leaflets of the valve.

Optionally, the prosthetic heart valve may comprise cuffs or other protective layers arranged between the stent and the leaflets and/or around the exterior of the stent.

In step s1002, the balloon is inflated such that it contacts two or more of the leaflets of the prosthetic heart valve. Preferably the balloon contacts all leaflets of the prosthetic heart valve.

Preferably, the balloon is inflated sufficiently to apply a radial force to the leaflets and to push the leaflets outwards towards the surrounding stent. Consequently, the leaflets may be flattened against the stent, or against any intervening layer between the leaflets and stent. This may place the leaflets in a fully open position. Alternatively, this flattening of the leaflets of the prosthetic heart valve relative to the surrounding stent may occur as the valve is subsequently crimped and its diameter reduced (see step s1003). The flattened arrangement of the leaflets is particularly uniform and consistent. Folds or inconsistencies in the leaflet arrangement may be minimised.

In step s1003, the prosthetic heart valve is crimped to reduce its external diameter and the external diameter of the balloon. The balloon is maintained in contact with the two or more leaflets during crimping as the diameter of the valve is reduced. As such, the balloon will apply radial and/or frictional forces to the leaflets and restricts the movement of the leaflets relative to the stent. Therefore, leaflets are supported by the balloon throughout crimping, and the balloon acts to restrict leaflets from bunching or folding irregularly, and especially restricts bunching or folding in the axial direction along the valve. Hence, the leaflets are formed in particularly consistent arrangement. This allows the valve to have a smaller final diameter (e.g. a smaller delivery diameter) than achieved using pre-existing approaches. Furthermore, the risk of damage to the leaflets during crimping is reduced.

As previously discussed, the crimping may involve multiple steps and can be performed using multiple balloons which are replaced between crimping steps. The internal pressure within a balloon may be varied or released entirely between or during crimping steps.

In further examples, the balloon may be inflated to a predetermined initial pressure, but not to a dimension in which it contacts two or more of the leaflets of a prosthetic heart valve. The prosthetic heart valve may then be crimped onto the inflated balloon such that the leaflets of the valve contact and are flattened as discussed above. Thereafter, crimping using an inflated balloon to support the leaflets may continue as discussed above.

Following the preparation of a prosthetic heart valve using method 1000, the prosthetic heart valve may be delivered and implanted as discussed above with reference to FIGS. 4 to 11.

Support members used during crimping to support the leaflets of a prosthetic heart valve are not limited to the balloons discussed above. Alternative support members may include compressible bodies—e.g. a cylindrical foam or rubber body.

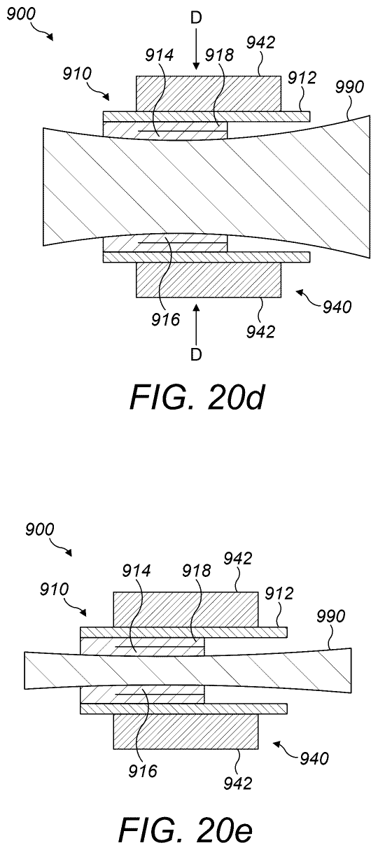

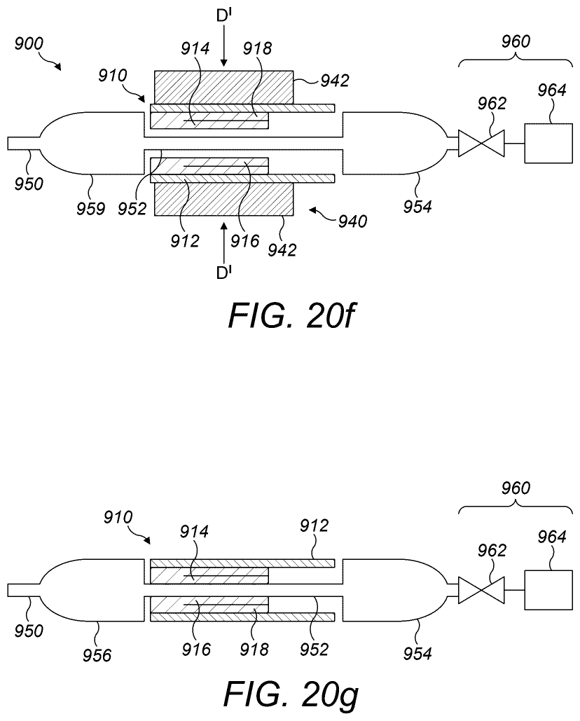

The use of support members generally and compressible bodies specifically will now be discussed with reference to FIGS. 19 and 20. FIG. 19 shows a flow chart diagram of a method 2000 for preparing an expandable prosthetic heart valve for delivery using a support member. Whilst FIGS. 20a to 20g show sequential schematic cross-sectional arrangements of a system 900 comprising a compressible body that performs a further method according to the present disclosure.

Returning to FIG. 19, it will be appreciated that the steps shown in this method 2000 are common to all approaches discussed herein, including both those that use a balloon and a compressible support member.

In step s2001, a support member (e.g. a balloon such as a sacrificial balloon or delivery balloon, or a compressible body) is inserted into a prosthetic heart valve whilst the prosthetic heart valve is in an expanded state. The prosthetic heart valve may comprise a stent, two or more leaflets and one or more cuffs or further layers. The support member is inserted into the aperture or lumen defined between the leaflets within the stent. The support member is preferably inserted entirely through the prosthetic heart valve. Where the support member is a balloon, it may be inserted in a deflated or partially inflated state. Where the support member is a compressible body, the compressible body may be compressed to a size that is smaller than the aperture between the leaflets of the expanded valve for insertion, although this is not essential.

In step s2002, the support member and prosthetic heart valve are arranged such that the support member contacts two or more leaflets of the prosthetic heart valve. If the support member is a balloon, it may be inflated such that it expands and contacts the leaflets of the valve. If the support member is a compressible body and the body was compressed during its insertion into the valve, the compressible body may be allowed to expand (i.e. uncompress) such that it contacts the leaflets of the valve. Alternatively, in an initial crimping step, the prosthetic valve may be partially crimped onto the exterior surface of the support body—e.g. the exterior surface of an inflated balloon or compressible body that have an external diameter that is less than the internal diameter of the valve in its expanded state. Preferably the system is arranged such that the support member contacts all of the leaflets of a valve, although this is not essential.

As discussed above with reference to the previous examples, as the support body contacts the leaflets it preferably applies a radial force to the leaflets and pushes the leaflets outwards towards the surrounding stent. This may flatten the leaflets against the stent (an intervening cuff or layer between the leaflets and stent) so that the leaflets are in a “fully-open” position.

Thereafter, in step s2003, the prosthetic heart valve is crimped to reduce its external diameter and the external diameter of the support member, wherein the support member is maintained in contact with the two or more leaflets during crimping. The support member applies radial and/or frictional forces to the leaflets to restrict the movement of the leaflets relative to the stent. Therefore, leaflets are supported throughout crimping, and the support member acts to prevent unintended bunching or folding of the leaflets.

As previously discussed, the crimping may involve multiple steps. The crimping method may be performed using multiple balloons which are replaced between crimping steps. Equally, the compressible body may be replaced with a balloon between crimping steps or after crimping.

This approach provides a crimped prosthetic heart valve that has an especially consistent, compact and uniform arrangement of leaflets. As such, the final valve diameter (e.g. a delivery diameter or crimp profile) achieved using this method can be made smaller than when using pre-existing approaches. In addition, the risk of damage to the leaflets during crimping is reduced, thereby increasing the performance and lifespan of the valves.

Following the preparation of a prosthetic heart valve using method 2000, the prosthetic heart valve may be delivered and implanted as discussed above with reference to FIGS. 4 to 11.

FIG. 20a to 20g show in schematic cross-section, sequential steps in a method of preparing a prosthetic heart valve 910 using a system 900 comprising a compressible body 990. The material of the compressible body 990 may be any suitable material preferably compressible down to at least 50% of its original size.

Preferably the compressible body 990 comprises one or more of polyurethane foam, polyethylene foam, polypropylene foam, polyester foam, and neoprene rubber foam. The compressible body 990 is able to maintain the relative position of the leaflets 914, 916 during crimping (as discussed below) but preferably remains sufficiently compressible such that it does not significantly inhibit crimping and does not impart so much force during crimping that it damages the leaflets 914, 916 by compression.

In the example shown in FIG. 20a, the compressible body 990 has an external diameter that is greater than the internal diameter of the aperture between the leaflets 914, 916 of the expanded valve 910, and optionally comprises an external diameter that is greater than the external diameter of the stent 912 when the valve 910 is expanded. However, in further examples, the dimensions of the compressible body 990 (the support element) would be approximately the dimensions of the parent diameter of the stent. Generally, the compressible body 990 may have an uncompressed external diameter that is at least 75% of the internal diameter of the valve 910 when the valve is in its expanded state (e.g. at least 80, 90 or 95% of the internal diameter of the internal diameter of the expanded valve 910). For example, the compressible body 990 may have an uncompressed external diameter that is greater or equal to the internal diameter of the expanded valve 910 plus 5 millimetres and/or less than or equal to the internal diameter of the expanded valve 910 minus 5 millimetres. In a particularly, preferred example the compressible body 990 may have an internal diameter that is equal to the internal diameter of the expanded valve 910 minus 1 millimetre. In examples where the compressible body 990 has an uncompressed external diameter that is greater or equal to the internal diameter of the expanded valve 910, the method may include inserting the compressible body 990 into the prosthetic heart valve 910 and crimping the valve 910 onto the outer surface of the compressible body 990 in an initial crimping step before performing additional crimping steps during which the leaflets 914, 916 are supported by the compressible body 990.

As shown in FIG. 20b, the compressible body 990 is compressed to a diameter that is smaller than the internal distance between the leaflets 914, 916 of the expanded valve 910, and inserted into the prosthetic heart valve 910. As illustrated, the compressible body 990 extends through the interior of the stent 912 and through the aperture between the leaflets 914, 916. The compressible body 990 may be compressed for this step manually or using a clamp, a band or a tube of the appropriate dimensions, or any other suitable device. Preferably the compressible body may be compressed and inserted into the prosthetic heart valve 910 manually.

After insertion into the prosthetic heart valve 910, the compressible body 990 is released and allowed to expand, as shown in FIG. 20c. While expanding, the exterior surface of compressible body 990 will contact the leaflets 914, 916 of the prosthetic heart valve 910. The compressible body 990 applies a radial force to the leaflets 914, 916, and will preferably flatten the leaflets against the cuff 918 and stent 912. As such, the leaflets 914, 916 are placed in a fully-open arrangement. Hence, the compressible body 990 will act on the leaflets 914, 916 in a similar manner to the balloons discussed above. The arrangement of the valve 910 and leaflets 914, 916 in FIG. 20c is similar to those shown in FIGS. 13b and 14a.

Thereafter, the valve 910 and compressible body 990 are inserted into a crimper 940 as seen in FIG. 20d. However, this is not essential and in alternative examples a valve 910 may be inserted into a crimper 940 before a compressible body 990 is inserted into the valve 910.

In a first crimping step, the crimper 940 is operated to close its jaws 942 and partially crimp the valve 910, as shown by arrows D. In this first crimping step, the external diameter of the prosthetic heart valve 910 is reduced from its expanded diameter to a support release diameter. The partially crimped prosthetic heart valve 910 is shown in FIG. 20e.

During this first crimping step, the compressible body 990 remains in contact with the leaflets 914, 916 of the valve 910, restricting their movement relative to the stent 912 and cuff 918. In particular, the leaflets 914, 916 are preferably prevented from bunching or folding in either the longitudinal or circumferential directions.

Following the first crimping step, the compressible body 990 is removed from within the valve 910 and replaced with a delivery balloon 950, as shown in FIG. 20f. The compressible body 990 may be compressed further using a clamp or other device before removal to avoid changing the positioning of the leaflets 914, 916. The delivery balloon 950 has the same features as the delivery balloon 550 discussed below with reference to FIGS. 14c to 14e. The delivery balloon 950 is connected to a pressure regulation system 960 which has the same features as the pressure regulation system 560 shown in FIGS. 14c to 14e. The pressure regulation system 960 comprises a pressure regulator 962 and a fluid supply device 964.

The valve 910 may be retained in the crimper 940 as the compressible body 990 is removed and the delivery balloon 950 inserted therein. However, this is not essential and in further examples, the valve 910 may be removed from the crimper for these steps and returned to the crimper afterwards.

Thereafter, in a second crimping step, the crimper 940 is operated to further close its jaws 942 and crimp the prosthetic heart valve 910 over and onto the delivery balloon 950 as shown by arrows D′ in FIG. 20f. In this step the external diameter of the prosthetic heart valve 910 may be reduced from the support release diameter to a delivery diameter.

As shown, the delivery balloon is deflated in the second crimping step to avoid affecting proximal and distal pillowed portions 954, 956. However, this is not essential and in further examples the delivery balloon 950 may be inflated such that it contacts the leaflets 914, 916 of the valve 910 during the second crimping step. The pillowed portions 954, 946 of the delivery balloon 950 may be constrained using sheaths or other securing members as previously discussed.

As shown in FIG. 20g, the prepared assembly comprising the delivery balloon 950 and crimped prosthetic heart valve 910 may then be removed from the crimper 940. The resulting crimped prosthetic heart valve 910 comprises leaflets 914, 916 that are provided in a particularly compact and consistent arrangement.

The prosthetic heart valve 910 may be collapsed (crimped) to a smaller diameter than if no support member was used. Moreover, damage to the leaflets from folds and wrinkles during crimping is reduced. The delivery balloon 950 may subsequently be used to deliver and implant the valve 910 into a patient as previously discussed.

The method discussed above with reference to FIGS. 20a to 20g is particularly suited for prosthetic heart valves having an external diameter in their full expanded state in the range from 15 to 50 mm, and preferably from 18 to 30 mm, and for crimping such valves to a final or delivery diameter in the range from 4 to 12 mm, preferably in the range from 5 to 10 mm. The support release diameter may be in the in the range from 8 to 20 mm, and is preferably in the range from 10 to 16 mm.

The external diameter of the prosthetic heart valve in its expanded state may be in the range from 2 to 5 times the delivery diameter. Whilst the support release diameter may be in the range from 1.1 to 2 times the delivery diameter.

Where the compressible body 990 comprises an external diameter that is less than the internal diameter of the prosthetic heart valve 910 in its expanded state, the first crimping step may involve an initial phase where the valve is initially crimped (collapsed) such that its leaflets 914, 916 contact the exterior surface of the compressible body 990. Alternatively, this process of arranging the leaflets 914, 916 of the valve 910 so that they contact the compressible body 990 may occur before the first crimping step.

As discussed above, the use of a support member such as a balloon or compressible body acts to place the leaflets in a uniform, consistent arrangement before crimping and maintains the leaflets in this arrangement during crimping.

Consequently, a prosthetic heart valve may be crimped to a smaller diameter and is less likely to sustain damage to its leaflets during crimping.

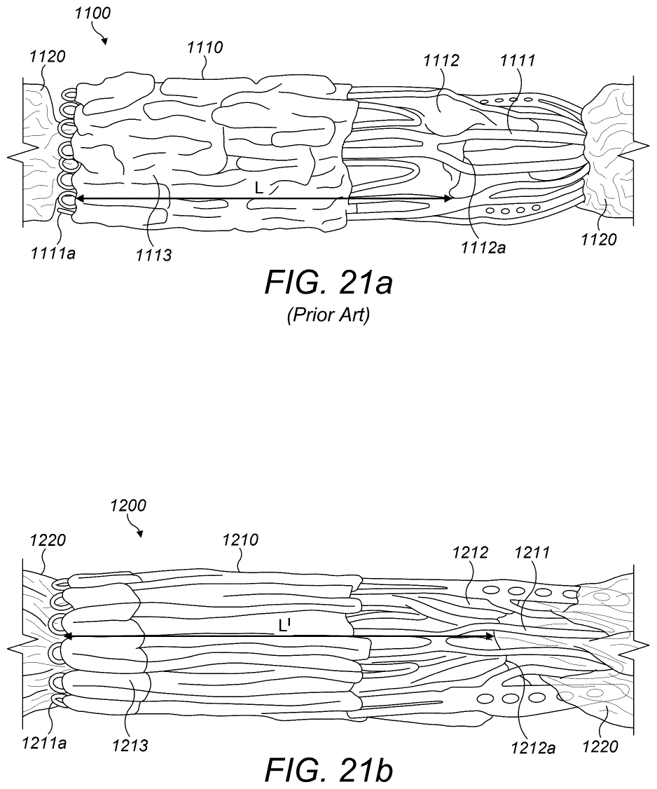

FIGS. 21a and 21b offer a comparison between two prepared prosthetic heart valve delivery assemblies 1100, 1200 with similar components. In each case, a prosthetic heart valve 1110, 1210 is crimped onto a balloon 1120, 1220 (e.g. a delivery balloon). Each prosthetic heart valve 1110, 1210 had an expanded diameter of 29 mm before crimping, and is crimped to a final (delivery) diameter of approximately 7 to 8 mm. More generally, the methods discussed herein are well suited for crimping prosthetic heart valves to have external diameters in the range from 5 to 10 millimetres.

The first, prior art assembly 1100 shown in FIG. 21a comprises a prosthetic heart valve 1110 that has been crimped onto a balloon 1120 whilst the balloon 1120 remained deflated. The prosthetic heart valve 1110 comprises a stent 1111, three leaflets 1112 arranged inside the stent 1111, and an outer cuff 1113 arranged around the stent 1111. The leaflets 1112 and outer cuff 1113 are attached to the stent 1111 at its distal end to the left of the drawing. The prosthetic heart valve 1110 also includes an inner cuff, which is not visible in the view of FIG. 21a. Specifically, the leaflets 1112 are attached to the inner cuff, the inner cuff being attached to eyelets 1111a at the distal end of the stent 1111.

In contrast, the second assembly 1200 comprises a prosthetic heart valve 1210 that is crimped over a balloon 1220 using the methods discussed above, where the balloon was inflated such that it contacted and supported the leaflets 1212 of the valve 1210 during crimping. Again, the prosthetic heart valve 1210 of this assembly 1200 comprises a stent 1211, three leaflets 1212 arranged inside the stent 1211, and an outer cuff 1213 arranged around the stent 1211. The prosthetic heart valve 1210 also includes an inner cuff, which is not visible in the view of FIG. 21b. The leaflets 1212 and outer cuff 1213 are attached to the stent 1211 at its distal or inflow end 1211a to the left of the drawing.

Specifically, before crimping of the second assembly 1200, the balloon 1220 was inflated to an internal pressure of 20.7 kPa (approximately 3 psi or 0.2 atm) over atmospheric pressure such that the balloon 1220 contacted the leaflets 1212 of the valve 1210. The internal pressure within the balloon 1220 was maintained at 20.7 kPa (approximately 3 psi or 0.2 atm) using a syringe with a pressure gauge as the prosthetic heart valve 1210 was crimped from its initial diameter of 29 mm to an intermediate diameter of approximately 15 mm. Thereafter the internal pressure within the balloon 1220 was released and the prosthetic heart valve 1210 was crimped further to its final (delivery) diameter of 8 mm. As such, the crimping involved two steps—a first crimping step where the balloon 1220 is inflated and supports the leaflets 1212 of the valve 1210 as the valve 1210 is crimped to an intermediate diameter and a second crimping step in which the pressure within the balloon 1220 was released and the valve 1210 is crimped to a final or delivery diameter.

The leaflets 1112, 1212 of both assemblies 1100, 1200 each have a similar structure to the leaflet 90 shown in FIG. 3. The leaflets 1112, 1212 have an attached edge which is attached by suturing to the distal or inflow end of the stent 1111, 1211 below the corresponding outer cuff 1113, 1213. In addition, the leaflets have 1112, 1212 a free edge 1112a, 1212a which is visible through the cells of the collapsed stent 1111, 1211 towards the right side of the figures.

When comparing the two assemblies 1100, 1200 the arrangement of their leaflets 1112, 1212 are noticeably different. The leaflets 1112 of the first assembly 1100 are more irregular and contain significant wrinkles and folds perpendicular to the length of the valve 1110 and stent 1111 (i.e. from right to left as shown). The free edges 1112a of the leaflets 1112 are bunched towards the distal or inflow end of the stent 1111 where the leaflets 1111 are attached. Significant folds of leaflet tissue extend outwards from the valve 1110 through cells in the stent 1111. In contrast, the leaflets 1212 of the second assembly 1200 have a more uniform arrangement. Notably, the leaflets 1212 have not been folded or moved significantly in the axial direction along the valve 1210 (i.e. from right to left as shown). This provides the benefit that the leaflets 1212 of the valve 1210 that was supported during crimping is more evenly distributed across the length of the assembly 1200, decreasing the bulk and diameter of the assembly 1200, especially in the region covered by the cuff 1213. Furthermore, the folds within the leaflets 1212 are smaller and more consistent, reducing local tissue compaction which can lead to calcification. The folds in the leaflets 1212 of the valve 1210 that was supported during crimping extend a smaller distance through the cells in the stent 1211 and as such are less prone to damage during delivery.

The bunching of leaflets 1112, 1212 along the valves 1110, 1210 may be compared using the lengths L, L′ marked on FIGS. 21a and 21b. This length L is marked between the run of eyelets 1111a, 1211a at the distal or inflow end of the stent 1111, 1211 and the nearest part of the free edge 1112a, 1212a of the leaflets 1112, 1212. The eyelets 1111a, 1211a of the stent 1111, 1211 offer a reference point for the position of the attached edge of the leaflets 1112, 1212, because the part of the attached edge that is arranged furthest in the distal or inflow direction is sutured to or close to these eyelets 1111a, 1211a.

As seen, the length L of FIG. 21a is significantly smaller than the length L′ of FIG. 21b. This indicates that significantly less undesirable bunching is present in the assembly 1200 in which the valve 1210 was supported throughout crimping.

The lengths L and L′ marked on FIGS. 21a and 21b are indicative of the minimum distance between the free edge 1112a, 1212a of each leaflet 1112, 1212 and a reference point on the attached edge of each leaflet 1112, 1212 that is arranged furthest from the free edge 1112a, 1212a in the distal or inflow direction. This minimum distance is the distance between the point on the free edge 1112a, 1212a that is closest to said reference point on the attached edge. As the relative positions of the eyelets 1111a, 1211a and the reference point on the attached edge of the leaflets 1112, 1212 are known from manufacture, the minimum distance can be calculated from length L and L′.

In preferred examples, expandable prosthetic heart valves are crimped so that the leaflets are arranged such that, for each leaflet, the distance between a reference point on the attached edge, the reference point being the point on the attached edge which is furthest from the free edge along a central axis of the stent (e.g. a position at or close to the eyelets 1111a, 1211a shown on FIGS. 21a and 21b), and a point on the free edge which is nearest to the reference point along a direction parallel to a central axis of the stent is at least 80% of a nominal length of the leaflet, the nominal length of the leaflet being the maximum distance between the attached edge of the leaflet and the free edge of the leaflet along a direction parallel to a centreline of the leaflet when the leaflet is laid flat (e.g. the length H₀ shown in FIG. 3). Preferably this distance is at least 90% of the nominal length of the leaflet, more preferably at least 95%. Such an arrangement is exhibited by the assembly 1200 shown in FIG. 21a. This arrangement is not easily achieved using approaches where a valve is crimped without internal support.

Other measures of the relative bunching of leaflets within crimped prosthetic heart valves are also possible. For instance, the distance between the free edge of each leaflet and the end of the stent (e.g. the proximal or outflow end, to the right in FIGS. 21a to b) to which the leaflets are not attached may be measured.

In addition, if the first and second assemblies 1100, 1200 shown in FIGS. 21a and 21b respectively were to be cut perpendicular to the axis of the stent (i.e. perpendicular to the distal-proximal direction) to reveal their cross-sections, it would be seen that the leaflets 1212 of the second assembly 1200 are interleaved between the material of the corresponding balloon 1220 in a circumferential direction as the two components have been compressed together. Whereas it would be observed that the leaflets 1112 of the first assembly 1100 are folded onto the surface of the respective balloon 1120 because the balloon 1120 of the first assembly 1100 remained deflated throughout the crimping process. The interleaving of the leaflets 1212 and the balloon material in the second assembly 1200 also enables the respective valve 1210 to be more tightly crimped relative to the valve 1110 in the first assembly 1100. A similar interleaved arrangement of the leaflets and balloon is possible with all of the methods discussed above where a balloon is in contact with the leaflets as they are crimped (e.g. where a balloon or delivery balloon is inflated whilst a valve is crimped).

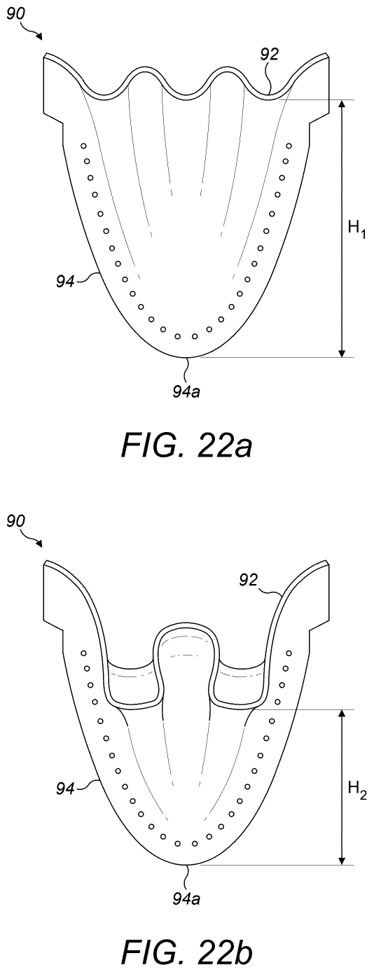

The arrangement of leaflets in valves that have been crimped with and without support will be described further with reference to FIGS. 22a and 22b. FIGS. 22a and 22b show two alternative arrangements of a leaflet 90 similar to the one described above with reference to FIG. 3. In FIGS. 22a and 22b, the leaflet 90 is shown without the other components of the valve to aid understanding, although in practice the leaflet 90 will be attached to other components of the valves discussed above.

Components shown in FIGS. 22a and 22b that have corresponding counterparts in FIG. 3 are denoted with the same reference signs. These components may have any of the features and benefits discussed above. The leaflet 90 has a concave free edge 92 configured to cooperate with the free edges of other leaflets to help provide the one-way valve functionality, an attached edge 94 by which it may be attached to a stent and/or cuff (skirt) using a plurality of holes 98. The leaflet 90 comprises leaflet tabs 96 by which the leaflet 90 may be attached to adjacent leaflets.

FIG. 22a shows the leaflet 90 in a uniform, regular arrangement achieved when crimping using an internal support body (e.g. an inflated balloon or a compressible body) as discussed above. For instance, the leaflets 1212 of the assembly 1220 shown in FIG. 21b will exhibit a similar shape and arrangement to the leaflet 90 shown in FIG. 22a. The leaflet 90 comprises a plurality of relatively small and regular “accordion-like” folds arranged along the length of the leaflet 90 (e.g. top to bottom as shown in FIG. 22a and parallel to the axis of a valve or stent in which the leaflet is positioned). There is little bunching in the axial or longitudinal direction (top to bottom of FIG. 22a). The distance H₁ between a reference point 94a on the attached edge 94 which is furthest from the free edge 92 along a centreline of the leaflet 90 (and along the central axis of any surrounding stent and valve) and the closest part of the free edge 92 is relatively large and is preferably at least 80%, preferably 90% and more preferably 95% of the nominal length H₀ of the leaflet 90 (shown in FIG. 3). Hence, the material of the leaflet 90 shown in FIG. 22a is evenly distributed along its length. For these reasons, the maximum size of a valve can be reduced and the risk of damage to leaflets during crimping and/or during delivery is minimised when crimping using the support bodies discussed above.

FIG. 22b shows the leaflet 90 in an irregular arrangement that is typical of crimping without any internal support—e.g. when a valve is crimped around an uninflated or deflated balloon. The leaflet 90 forms fewer folds along the length of the leaflet (top to bottom as shown in FIG. 22a), and is significantly bunched, with the free edge 92 being folded over toward the attached edge 94. The distance H2 between the reference point 94a on the attached edge 94 which is furthest from the free edge 92 along a centreline of the leaflet 90 (parallel to the central axis of any surrounding stent and valve) and the closest part of the free edge 92 is relatively small and is less than 80% of the nominal length H0 of the leaflet 90 (shown in FIG. 3). In a valve that has been crimped without support and includes leaflets with the arrangements similar to that shown in FIG. 22b the large, irregular folds will increase the size of the valve. Furthermore, the bunching in the longitudinal direction between the free edge 92 and attached edge 94 which creates folds perpendicular to the length of a valve can cause the material of the leaflet 90 to be compacted locally which can lead to calcification.

The difference between the consistent, regular arrangement of folds in leaflets 90 achieved when crimping prosthetic valves with internal support (as shown in FIG. 22a) when compared to the irregular arrangement typical when crimping without support (FIG. 22b) is reflected in how the leaflets interact with the cells of the surrounding stent.

In a valve or valve assembly crimped with internal support (e.g. with leaflets 90 similar to the example shown in FIG. 22a) the material or tissue of the leaflet 90 is consistently folded and will not project a significant distance through any cells in an overlying stent because each longitudinal fold is relatively small. That is, any portions of the leaflet 90 which extend through the cells of the surrounding stent will extend beyond the struts of the stent by less than 20% of the outer diameter of the crimped prosthetic heart valve, and preferably less than 15% or 10%. These distances may be measured on a prepared prosthetic heart valve assembly using a vision system such as a microscope or camera, or using any other suitable measuring device.

In contrast, in valves that have been crimped without internal support with leaflets (as shown in FIG. 22b) the relatively large irregular folds formed in the leaflets of valves crimped without internal support will tend to project significantly through the cells of the stent (e.g. by greater than 20% of the outer diameter of the crimped prosthetic heart valve). These folds which extend beyond the stent by a significant distance are at risk of damage as the valve is manipulated during delivery.

In addition, it is possible to distinguish between prosthetic valves and assemblies that have been crimped with internal support using the methods discussed above and those that have not, using the proportion of cells into which, or through which, longitudinal folds of leaflet material extend. Such longitudinal folds extend substantially parallel to the centreline of their stent or valve (i.e. with an angle that is less than 45 degrees from the centreline of the stent or valve).

Where valves have been crimped with internal support, the leaflets will generally be arranged such that either a single fold of the leaflet material or two folds of leaflet material extends into or through each cell in at least one row of the stent. As an example, a single fold of the leaflet material or two folds of leaflet material may extend into or through each cell in the row of cells that is furthest from the attached edges of the leaflet(s)—e.g. into and through each cell 34 in the outflow-most row of cells in the valve 10 and stent 20 discussed above in reference to FIGS. 1 to 4.

For example, at least 75% of the cells in the row of cells furthest from the attached edges of the leaflets (e.g. the outflow-most row of cells) may receive two longitudinal folds of leaflet material therein, preferably at least 80%, more preferably at least 90%, more preferably still all of the cells receive two longitudinal folds of leaflet material therein. Alternatively, at least 75% of the cells in the row of cells furthest from the attached edges of the leaflets (e.g. the outflow-most row of cells) may receive a single respective longitudinal fold of leaflet material therein, preferably at least 80%, more preferably at least 90%, more preferably still all of the cells receive a single respective longitudinal fold of leaflet material therein.

These consistent arrangements of longitudinal folds of leaflet material may be observed throughout valves that have been crimped with internal support. None, or very few, of said cells have no longitudinal folds or larger numbers of longitudinal folds extending into or through them when valves are crimped using internal support as discussed above. The consistent arrangements help reduce the overall size of the valves and minimises damage to the leaflets. In contrast, in prosthetic heart valves and assemblies that have been crimped without internal support, the leaflets comprise irregular folds, so that for some of the outflow-most cells there may be three or more longitudinal folds of leaflet that extend into or through them, and in other cells there may be no longitudinal folds present at all.

As such, in valves that have been crimped without support, a lower proportion of the cells in a given row of cells (e.g. the outflow-most row of cells, or the row of cells furthest from the attached edges of the leaflets) will receive a single longitudinal fold or two longitudinal folds of leaflet material that extends into or through them.

In more detail, a consistent arrangement of leaflets can be obtained using internal support when crimping valves that comprise stents (frames) with an inflow section comprising a plurality of rows of first cells and an outflow or hybrid section comprising a row of larger second cells, the second cells defining a larger internal area, height and/or width than the first cells. An example of such valves and stents are discussed with reference FIGS. 1 to 4 above, and a portion of such a stent is shown especially clearly in FIG. 2. As shown in FIG. 2, the cells 30, 32 of the inflow section 22 may be hexagonal. However, in other examples they may have other shapes, being diamond-shaped, chevron-shaped, or other suitable shapes. When these valves are crimped using internal support as discussed above, a single longitudinal fold of leaflet material will tend to enter each first cell in the outflow-most row of the inflow section, whilst two longitudinal folds of leaflet material will tend to extend through and beyond each second cell in the outflow section. For example, at least 75% of the cells in the outflow-most row of first cells of the inflow section (the row of cells in the inflow section furthest from the attached edge of the leaflets) receive a single respective longitudinal fold of leaflet material therein, preferably at least 80%, more preferably at least 90%. These folds of leaflet material may enter but not extend significantly through the first cells.

Additionally, or alternatively, at least 75% of the second cells in the outflow section receive two longitudinal folds of leaflet material therein, preferably at least 80%, more preferably at least 90%. By comparison these folds tend to extend full through the larger second cells which are arranged further from the attached edge of each leaflet.

In contrast, when valves with the stents (frames) of FIG. 2 are crimped without internal support, the relationship between folds of leaflet material and the cells of the stent are much more irregular. There may be relatively large numbers of first cells in the outflow-most row of cells of the inflow section into which no longitudinal folds or two or more longitudinal folds of leaflet material extent, and/or relatively large numbers of second cells in the outflow section through which no longitudinal folds, one longitudinal fold or three or more longitudinal folds protrude.

A further means of distinguishing between prosthetic valves which have been crimped with internal support in accordance with the invention and those that have been crimped without internal support is the variance or variation in the position of the free edge of the leaflets in their crimped state.

For example, a variance σ² in the position of the free edges of the leaflets of a valve may be calculated by obtaining a plurality of measurements x₁ to xₙ of the distance parallel to the centerline of the valve between the free edges of the leaflets in a valve and a consistent point on the valve or valve assembly (e.g. the eyelets 1111a shown in FIGS. 21a and 21b, the extreme point of the attachment edge, or an end of the stent), and using the calculation:

$${σ}^{2} = \frac{{\sum }_{i = 1}^{n} {( {x}_{i} - \overline{x} )}^{2}}{N}$$

where xᵢ are the measured distances, \tilde{x} is the mean of the measured distances and N is the number of measurements in the date set. It will be noted that since the mean distance \tilde{x} is subtracted from each measurement in the summation that the resulting variance is dependent only on the variation in the leaflet height and not the location of the consistent point from which each of the distances x₁ are measured. In other words, the position of said point on the valve does not affect the measurement of the variance so long as the same point along the longitudinal length of the valve (parallel to the centreline of the valve and stent) is used for each measurement.

Preferably multiple measurements are obtained between the free edge of each leaflet and the consistent point. For instance, three or four measurements may be obtained for each leaflet, such that in a valve with three leaflets the variance may be based on nine or twelve measurements. The measurements may be obtained at predetermined positions on the valve, for example, at a consistent spacing around the circumference of the valve (e.g. every 30 degrees around the circumference). Alternatively, the measurements may be taken at predefined points relative to the stent or other components of the valve. For example, the distances may be measured from one or more points on the free edge of the leaflets that are immediately adjacent to the struts of the stent—e.g. where each free edge passes under a strut of the stent—and/or from points on the free edge of the leaflets that are at the centre or on the centreline of cells of the stent. However, in further examples distances may be taken from a random selection of positions around the valve. Where the free edge is folded such that there are two points on the free edge a leaflet that coincide with the intended measuring point—e.g. the free edge of the leaflet is folded under a struct of the stent twice—the measurements may be taken from either the point on the free edge that is nearest or furthest to the attached edge of the leaflet, so long as the choice is consistent within each valve and between the different valves being compared.

Crimping prosthetic valves with internal support from an inflated balloon, compressible body or other support member provide prosthetic valves with variances σ² in the position of the free edge of their leaflets of less than or equal to 1.2, preferably less than or equal to 1.0, more preferably less than or equal to 0.9 and more preferably still less than 0.8. These figures are applicable to a wide range of prosthetic heart valves having a range of final crimp diameters and numbers of leaflets, and using a wide variety of measurement positions. In comparison, prosthetic valves which are crimped without internal support (e.g. onto a deflated balloon) tend to have significantly higher variances σ²

Testing was performed for prosthetic heart valves having three leaflets and a stent with a similar arrangement to the example shown in FIG. 1 and FIG. 2, and with an uncrimped (expanded) diameter of 29 and a crimped (delivery) diameter in the range from 7.7 mm to 8.5 mm. When crimping using an internal support of a compressible foam body the valves had an average variance 62 of 0.77, as measured across 6 valves. Whereas when crimping without any support, valves had an average variance σ² of 1.76 across 11 valves. During this testing the variances σ² for each valve were calculated from 12 measurements, with four measurements being obtained from the free edge of each leaflet. For each leaflet, two measurements were taken from the points on the free edge of the leaflet at centre of each of the overlying outflow-most cells of the stent (large cells 34 as shown in uncrimped form in FIG. 2) and two measurements were taken at the points at which the leaflet passes from the overlying outflow-most cells under the frame of the stent towards the centre of the leaflet (e.g. where the leaflets pass under the first linking strut 35a or the adjoining upper strut 29 of each of the overlying outflow-most cells 34 as shown in uncrimped form in FIG. 2).

Alternatively, the arrangement of the leaflets in each valve may be quantified from a comparison of the difference between the average distance between the free edge of each leaflet and the point on the attached edge of each leaflet which is furthest from the free edge along a central axis of the stent and the minimum distance between the free edge of each leaflets and the point on the attached edge of each leaflet which is furthest from the free edge along a central axis of the stent (distances L and L′ shown in FIGS. 21a and 21b). Crimping prosthetic valves with internal support from an inflated balloon, compressible body or other support member offers prosthetic heart valves with less than 10% difference between said minimum distance and said average distance and preferably less than 8%, whereas this difference is greater for valves crimped without support. This difference V can be calculated for each valve as follows:

$$V = \frac{\overline{x} - {x}_{minimum}}{\overline{x}}$$

where \tilde{x} is the average distance between the free edge of each leaflet and the point on the attached edge of each leaflet which is furthest from the free edge along a central axis of the stent and xₘᵢₙᵢₘᵤₘ is the minimum distance between the free edge of each leaflets and the point on the attached edge of each leaflet which is furthest from the free edge along a central axis of the stent (distances L and L′ shown in FIGS. 21a and 21b). The average distance between the free edge of each leaflet \tilde{x} may be calculated from a plurality of measurements from respective points along the free edge as discussed above with reference to the calculations for variance.

In further examples, the proportion of each valve that is visible above any cuff (skirt) may also be used to assess whether the leaflets of the valves are consistently folded as discussed above.

In general, the arrangement of leaflets in a series of valves prepared with internal support in accordance with the methods described above will be significantly more consistent that a series of valves manufactured using traditional approaches without internal support.

For example, for a series of valves prepared using internal support in accordance with the methods described above—e.g. a series of sequentially or consecutively prepared valves or a sample of a series prepared valves taken from a larger series—the average of the variances σ² for each valve calculated using the methods described above may be less than or equal to 1.0, preferably less than or equal to 0.9 and more preferably less than 0.8. Whereas, the average of the variances σ² for a series of valves crimped without internal support is typically greater than 1.5. Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of preparing an expandable prosthetic heart valve comprising a stent and a plurality of leaflets for delivery, the method comprising the steps of:
inserting a support member into the prosthetic heart valve whilst the prosthetic heart valve is in an expanded state;
arranging the support member and prosthetic heart valve such that the support member contacts two or more leaflets of the plurality of leaflets of the prosthetic heart valve; and,
crimping the prosthetic heart valve to reduce its external diameter and the external diameter of the support member;
wherein the support member is maintained in contact with the two or more leaflets during crimping such that the support member applies a frictional or radial force to the two or more leaflets as the external diameter of the prosthetic heart valve is reduced.

2. A method according to claim 1, wherein arranging the support member and prosthetic heart valve such that the support member contacts the two or more leaflets of the prosthetic heart valve comprises arranging the support member to apply the radial force to the two or more leaflets that pushes said leaflets outwards towards the stent.

3. A method according to claim 1, wherein the support member is configured to restrict movement of the two or more leaflets relative to the stent as the external diameter of the prosthetic heart valve is reduced.

4. A method according to claim 3, wherein the support member is configured to restrict circumferential and/or longitudinal movement of the two or more leaflets relative to the stent as the external diameter of the prosthetic heart valve is reduced.

5. A method according to claim 1, wherein arranging the support member and prosthetic heart valve such that the support member contacts the two or more leaflets of the prosthetic heart valve comprises arranging the support member such that its external diameter is greater than 80% of the internal diameter of the stent when the stent is in the expanded state.

6. A method according to claim 1, wherein the support member is a balloon.

7. A method according to claim 6, wherein the balloon is inserted into the prosthetic heart valve while the balloon is in a deflated or partially inflated state.

8. A method according to claim 6, wherein:
arranging the support member and prosthetic heart valve such that the support member contacts the two or more leaflets of the prosthetic heart valve comprises inflating the balloon to a crimping pressure such that it contacts the two or more leaflets of the prosthetic heart valve;
and wherein the method further comprises:
maintaining sufficient internal pressure within the balloon during crimping in order to maintain the balloon in contact with the two or more leaflets.

9. A method according to claim 1, wherein crimping the prosthetic heart valve comprises partially crimping the prosthetic heart valve to reduce its external diameter to a support release diameter;
and wherein the method comprises the further steps of:
removing the support member from inside the partially crimped prosthetic heart valve; and
crimping the prosthetic heart valve further to reduce its external diameter from the support release diameter to a delivery diameter.

10. A method according to claim 1, wherein the balloon is a sacrificial balloon, and wherein crimping the prosthetic heart valve comprises partially crimping the prosthetic heart valve to reduce its external diameter to a support release diameter, and the method comprises the further steps of:
removing the sacrificial balloon from inside the partially crimped prosthetic heart valve;
inserting a delivery balloon into the prosthetic heart valve; and
crimping the prosthetic heart valve further to reduce its external diameter to a delivery diameter.

11. A method according to claim 10, wherein removing the sacrificial balloon from inside the partially crimped prosthetic heart valve comprises deflating the sacrificial balloon.

12. A method according to claim 10, further comprising:
inflating the delivery balloon such that it contacts two or more leaflets of the plurality of leaflets of the prosthetic heart valve; and,
maintaining sufficient internal pressure within the delivery balloon during crimping in order to maintain the delivery balloon in contact with said two or more leaflets.

13. A method according to claim 6, wherein a pressure within the balloon is maintained at a first predetermined crimping pressure during crimping.

14. A method according to claim 13, wherein the first predetermined crimping pressure is in the range from 0 to 203 kPa above atmospheric pressure.

15. A method according to claim 10, wherein during the step of crimping the prosthetic heart valve further to reduce its external diameter to a delivery diameter, the pressure within the delivery balloon is maintained at a second predetermined crimping pressure.

16. A method according to claim 15, wherein the second predetermined crimping pressure is in the range from 0 to 203 kPa above atmospheric pressure.

17. A method according to claim 6, wherein the pressure within the balloon and/or delivery balloon is controlled using a pressure regulator.

18. A method according to claim 17, wherein the pressure regulator comprises one or more of: a syringe; a manual syringe; a pressure gauge; a pressure relief valve; or a pump.

19. A method according to claim 6, wherein the method comprises:
partially crimping the prosthetic heart valve to reduce its external diameter to an intermediate diameter while sufficient internal pressure within the balloon is maintained in order to maintain the delivery balloon in contact with the two or more leaflets;
releasing the internal pressure within the balloon; and
crimping the prosthetic heart valve further to reduce its external diameter from the intermediate diameter to a delivery diameter.

20. A method according to claim 8, wherein the method comprises:
partially crimping the prosthetic heart valve to reduce its external diameter from the support release diameter to an intermediate diameter while sufficient internal pressure within the delivery balloon is maintained in order to maintain the balloon in contact with the two or more leaflets;
releasing the internal pressure within the delivery balloon;
crimping the prosthetic heart valve further to reduce its external diameter from the intermediate diameter to a delivery diameter.
